SEQUENCE LISTING (4 sequences)

SEQ ID NO: 1 - RNA, 22 nt, Homo sapiens
  source 1..22: mol_type = other RNA
  ncRNA 1..22: ncRNA_class = miRNA
tgaggtagtaggttgtgtggtt

SEQ ID NO: 2 - RNA, 23 nt, Homo sapiens
  source 1..23: mol_type = other RNA
  ncRNA 1..23: ncRNA_class = miRNA
taagtgcttccatgttttagtag

SEQ ID NO: 3 - RNA, 22 nt, Homo sapiens
  source 1..22: mol_type = other RNA
  ncRNA 1..22: ncRNA_class = miRNA
ggattcctggaaatactgttct

SEQ ID NO: 4 - RNA, 22 nt, Homo sapiens
  source 1..22: mol_type = other RNA
  ncRNA 1..22: ncRNA_class = miRNA
actttaacatggaagtgctttc

(19) United States
(12) Patent Application Publication
(10) Pub. No.: US 2023/0321133 A1
(43) Pub. Date: Oct. 12, 2023

(54) COMPOSITIONS AND METHODS FOR TREATING BACTERIAL INFECTIONS

(71) Applicant: THE TRUSTEES OF DARTMOUTH COLLEGE, Hanover, NH (US)

(72) Inventor: Bruce A. STANTON, Hanover, NH (US)

(21) Appl. No.: 17/961,024

(22) Filed: Oct. 6, 2022

Related U.S. Application Data
(63) Continuation-in-part of application No. PCT/US2021/025877, filed on Apr. 6, 2021.
(60) Provisional application No. 63/005,561, filed on Apr. 6, 2020.

(51) Int. Cl. A61K 31/7105; A61K 35/28; A61K 31/426; A61K 31/43; A61K 9/127; A61P 31/04; A61P 11/00

(52) U.S. Cl. CPC A61K 31/7105; A61K 35/28; A61K 31/426; A61P 11/00; A61K 9/127; A61P 31/04; A61K 31/43

(57) ABSTRACT

Compositions and methods for treating infectious disease. The composition includes a particle selected from the group consisting of liposome, extracellular vesicle, solid lipid nanoparticles, and polymeric nanoparticles; a miRNA; and an antibiotic, wherein the particle is loaded with at least one of the miRNA and the antibiotic.

### CROSS-REFERENCE TO RELATED APPLICATIONS

This application is a continuation-in-part of PCT/US2021/025877, filed on Apr. 6, 2021, which claims benefit of priority to U.S. Provisional Patent Application No. 63/005,561 filed on Apr. 6, 2020, the contents of which are incorporated herein by reference in their entirety.

### GOVERNMENT RIGHTS

This invention was made with government support under grant numbers NIH-R01 HL074175 awarded by the National Institutes of Health. The government has certain rights in the invention.

### BACKGROUND

P. aeruginosa infects about 51,000 patients every year in the US, and was 15 responsible for 2,700 deaths in 2017 alone, primarily due to antibiotic resistant strains of Pseudomonas aeruginosa¹. P. aeruginosa is an opportunistic pathogen that infects the lungs of immunocompromised individuals, including those with Chronic Obstructive Pulmonary Disease (COPD), Cystic Fibrosis (CF) and is an important cause of acute pneumonia and infection in burn wounds²⁻⁷. P. aeruginosa is one of the leading causes of nosocomial infections throughout the world, and ventilator associated pneumonia mortality caused by P. aeruginosa can be as high as 30% in some institutions². P. aeruginosa contributes to 5-10% of the acute exacerbations in COPD, which afflicts 24 million Americans, and is the 3^{rd} leading cause of death in the US^{5, 6, 8}. P. aeruginosa also chronically colonizes the lungs of about 50% of adults with CF, and its presence is strongly associated with reduced forced expiratory volume (FEW and progressive loss of lung function⁹⁻¹³.

Standard treatment for P. aeruginosa lung infections involves inhaled antibiotics, however, aerosolized antibiotics do not effectively permeate mucus or bacterial biofilms and they are also rapidly cleared from the lungs. Although antibiotics are an important part of lung disease management and have been shown to improve patient outcomes and reduce exacerbations, antibiotics do not decrease the abundance of P. aeruginosa or other co-infecting microbes effectively^{10, 11, 13}. This observation may be due to the development of antibiotic resistant strains of P. aeruginosa as a consequence of chronic antibiotic exposure that leads to upregulation of drug efflux pumps and β-lactamases¹⁴⁻²⁹. Moreover, P. aeruginosa forms biofilm, which are highly resistant to antibiotics. The World Health Organization (WHO) has identified P. aeruginosa as one of the ESKAPE pathogens (Enterococcus faecium, Staphylococcus aureus, Klebsiella pneumoniae, Acinetobacter baumannii, Pseudomonas aeruginosa, and Enterobacter species), which are responsible for the majority of nosocomial infections and are capable of “escaping” the biocidal action of antimicrobial agents²¹.

### SUMMARY

The present disclosure provides a composition comprising a) a particle selected from the group consisting of liposome, extracellular vesicle, solid lipid nanoparticles, and polymeric nanoparticles; b) a miRNA; and c) an antibiotic. In one embodiment, the particle is loaded with at least one of the miRNA and the antibiotic. In another embodiment, the particle is loaded with the miRNA. In another embodiment, the particle is loaded with the antibiotic. In another embodiment, the particle is loaded with both the miRNA and the antibiotic. In another embodiment, some particles are loaded with the miRNA, and some other particles are loaded with the antibiotic. In another embodiment, the miRNA and/or the antibiotic is encapsulated within the same particle. In another embodiment, the particles are made of materials that are non-antigenic. In another embodiment, the particles are made of materials that are non-allergenic.

In another embodiment, the particle is an extracellular vesicle. The vesicles may be isolated from a eukaryotic or a prokaryotic cell. Examples of eukaryotic cells are a mammalian cell, a yeast cell or a plant cell. An example of a prokaryotic cell is a bacterial cell. In another embodiment, the extracellular vesicle is derived from human cells. In another embodiment, the extracellular vesicle is derived from an adult stem cell. In another embodiment, the extracellular vesicle is derived from a mesenchymal stem cell. In yet another embodiment, the extracellular vesicle is derived from a human epithelial cell.

In another embodiment, the particle is a solid lipid nanoparticle. Solid lipid nanoparticles are formed of lipids that are solids at room temperature. In one embodiment, the solid lipid nanoparticles have an average diameter between 10 and 1000 nanometers. Suitable solid lipids include, but are not limited to, higher saturated alcohols, higher fatty acids, sphingolipids, synthetic esters, and mono-, di-, and triglycerides of higher saturated fatty acids. Solid lipids can include aliphatic alcohols having 10-40, preferably 12-30 carbon atoms, such as cetostearyl alcohol. Solid lipids can include higher fatty acids of 10-40, preferably 12-30 carbon atoms, such as stearic acid, palmitic acid, decanoic acid, and behenic acid. Solid lipids can include glycerides, including monoglycerides, diglycerides, and triglycerides, of higher saturated fatty acids having 10-40 or 12-30 carbon atoms, such as glyceryl monostearate, glycerol behenate, glycerol palmitostearate, glyceryl trilaurate, tricaprin, trilaurin, trimyristin, tripalmitin, tristearin, and hydrogenated castor oil. Suitable solid lipids can include cetyl palmitate, beeswax, or cyclodextrin.

In one embodiment, the miRNA targets an antibiotic efflux pump. In another embodiment, the miRNA targets a Resistance-Nodulation-Division (RND) drug efflux pump. In another embodiment, the miRNA targets a mexGHI-OpmD multidrug efflux pump. In another embodiment, the miRNA targets a biofilm gene in P. aeruginosa, Burkholderia cenocepacia, Streptococcus pneumoniae, or Staphylococcus aureus. In another embodiment, the miRNA opens pores or reduces protein abundance of at least one gene selected from the group consisting of NarG, NarH, NarI, NirS, NosZ, PvdA, PvdD, PvdH, PvdJ, PvdQ, PhzE1, and PhzE2a. In another embodiment, the miRNA targets β-lactamase. In another embodiment, the miRNA is let-7b having a sequence of ugagguaguagguugugugguu (SEQ ID NO: 1). In another embodiment, miRNA 302b-3p, miR-145-3p or miR-302b-5p having a sequence of uaagugcuuccauguuuuaguag (SEQ ID NO:2), ggauuccuggaaauacuguucu (SEQ ID NO:3) and acuuuaacauggaagugcuuuc (SEQ ID NO:4), respectively, target many of the same genes targeted by let-7b-5p.

In another embodiment, the antibiotic is selected from the group consisting of β-lactam antibiotics, fluoroquinolone antibiotics, and aminoglycoside antibiotics. In another embodiment, the antibiotic is selected from the group consisting of aztreonam, tobramycin, carbenicillin, azithromycin, colistin/polymyxin E, gentamicin and ciprofloxacin. In one embodiment, the amount of each of these antibiotics loaded onto the extracellular vesicle (EV) or nanoparticle (NP) is an amount of the antibiotic that, in conjunction with the microRNA (e.g., let-7b), is effective in inhibiting proliferation of Pseudomonas aeruginosa in the infected subject. In another embodiment, the amount of each of these antibiotics loaded onto the EV or NP is in the range of from about 1 μg to about 1 g per vesicle/particle. In another embodiment, the amount of each of these antibiotics loaded onto the EV or NP is in the range of from about 1 μg to about 1 mg per vesicle/particle. In another embodiment, the amount of each of these antibiotics loaded onto the EV or NP is in the range of from about 1 mg to about 100 mg per vesicle/particle.

In another embodiment, the composition is an inhalable powder, an aerosol, or a spray. In another embodiment, the composition is adapted for aerosolized administration. In another embodiment, the particle has a diameter ranging from 10 nm to 1,000 nm.

In an aspect, the present disclosure comprises a pharmaceutical composition comprising the compositions described herein and a pharmaceutically acceptable carrier.

In an embodiment, a kit comprises the pharmaceutical composition described herein and instructions for use.

In an embodiment, the compositions of the present disclosure are used in a method for inhibiting proliferation of biofilm-forming microorganisms. In an embodiment, the method comprises administering a therapeutically effective amount of the composition. In an embodiment, the biofilm-forming microorganisms are selected from the group consisting of Pseudomonas aeruginosa, Burkholderia cenocepacia, Streptococcus pneumoniae, or Staphylococcus aureus.

In an embodiment, the compositions of the present disclosure are used in a method for preventing or treating an infection associated with Pseudomonas aeruginosa in a subject in need thereof. In an embodiment, the method comprises administering to the subject a therapeutically effective amount of the composition.

In an embodiment, the compositions of the present disclosure are used in a method for reducing proliferation, survival, migration, or colony formation ability of a rapidly proliferating cell in a subject. In an embodiment, the method comprises contacting the cell with a therapeutically effective amount of the composition. In an embodiment, the cell is Pseudomonas aeruginosa, Burkholderia cenocepacia, Streptococcus pneumoniae, or Staphylococcus aureus.

In an embodiment, the compositions of the present disclosure are used in a method for reducing proliferation, survival, migration, or colony formation ability of a microorganism in a subject in need thereof. In an embodiment, the method comprises contacting the microorganism with a therapeutically effective amount of the composition. In an embodiment, the microorganism is Pseudomonas aeruginosa, Burkholderia cenocepacia, Streptococcus pneumoniae, or Staphylococcus aureus.

In an embodiment, the composition is administered in an aerosolized form.

In an embodiment, the composition is administered by inhalation.

In an aspect, the present disclosure comprises a method of preventing or treating an infection associated with Pseudomonas aeruginosa in a subject in need thereof. In an embodiment, the method comprises administering to the subject an extracellular vesicle loaded with let-7b in combination with an antibiotic. In an embodiment, the infection is a chronic lung infection. In an embodiment, the infection is a respiratory tract infection.

In an embodiment, the compositions of the present disclosure are used in a method for reducing or preventing biofilm formation by Pseudomonas aeruginosa on a living or nonliving surface. In an embodiment, the method comprises treating the surface with an effective amount of the composition.

In an embodiment, the compositions of the present disclosure are used in a method for preventing or treating an infection associated with a microorganism in a subject in need thereof. In an embodiment, the method comprises administering to the subject a therapeutically effective amount of the composition. In an embodiment, the microorganism is Pseudomonas aeruginosa, Burkholderia cenocepacia, Streptococcus pneumoniae, or Staphylococcus aureus. In an embodiment, the infection is a chronic lung infection. In an embodiment, the infection is a respiratory tract infection.

In an embodiment, the compositions of the present disclosure are used in a method for reducing or preventing biofilm formation by a microorganism on a living or nonliving surface. In an embodiment, the method comprises treating the surface with an effective amount of the composition. In an embodiment, the microorganism is Pseudomonas aeruginosa, Burkholderia cenocepacia, Streptococcus pneumoniae, or Staphylococcus aureus.

### BRIEF DESCRIPTION OF THE FIGURES

The following figures form part of the present specification and are included to further illustrate aspects of the present invention.

FIG. 1 shows that human bronchial epithelial cell (HBEC) EV increase fluoroquinolone sensitivity of P. aeruginosa. EV significantly decreased the planktonic growth of PA14 (OD600) in the presence of ciprofloxacin (CIP) (A), but did not affect growth in the absence of CIP (B). EV isolated from human bronchial epithelial cell cultures obtained from four wild type donor (WT-HBEC donors). ***p<0.001.

FIG. 2 shows that HBEC EV increase P. aeruginosa fluoroquinolone sensitivity by targeting MexGHI-OpmD. (A) EV decreased planktonic growth of PAO1 in the presence of CIP (0.03 μg/ml). Deletion of mexGHI-opmD (Δ ctrl) reduced planktonic growth of PAO1 to a similar degree as EV, and addition of EV had no effect on the knockout strain (Δ+EV). EV were isolated from six HBEC donors. ***p<0.001 and **p<0.01 vs. WT ctrl.

FIG. 3 shows that HBEC EV reduce biofilm formation by PA14 and increase the ability of beta-lactam antibiotics to reduce biofilm formation by PA14 and clinical isolates of P. aeruginosa. (A-F) EV significantly inhibited biofilm formation by P. aeruginosa strain PA14 (A) without significantly reducing planktonic growth of PA14 (B). EV significantly inhibited biofilm formation by P. aeruginosa strain PA14 in the presence of 0.1 μg/ml aztreonam (ATM, C) and 5 μg/ml carbenicillin (CAR, E). EV did not significantly reduce planktonic growth of PA14 in the presence of these sub-inhibitory concentrations of aztreonam (D) or carbenicillin (F). (G) EV significantly reduced biofilm formation in the presence of 20 μg/ml carbenicillin in four of six clinical isolates of P. aeruginosa. (H) EV did not significantly reduce planktonic growth of clinical isolates of P. aeruginosa in the presence of 20 μg/ml carbenicillin. Biofilms were measured using the crystal violet 96 well plate biofilm assay (OD550). Horizontal lines indicate means±SEM. A two-tailed unpaired Welch's t-test was used to calculate P values; N=3-6 biological replicates with EVs isolated from 3-6 human bronchial epithelial cell (HBEC) donors; ns=not significant.

FIG. 4 shows that HBEC EV increase the beta-lactam sensitivity of planktonic P. aeruginosa. (A) EVs decreased the minimal inhibitory concentration (MIC) of aztreonam (ATM) more than 2-fold (MIC=2.0 μg/ml in control vs. 0.8 μg/ml with EVs). (B) EV decreased the non-inhibitory concentration (NIC) of aztreonam (NIC=0.6 μg/ml in control vs. 0.1 μg/ml with EVs). (C and D) In the presence of 0.8 μg/ml aztreonam (about one-half the MIC) EV reduced planktonic growth of P. aeruginosa as determined by OD600 (C) as well as colony forming units (CFU, D). Horizontal lines indicate means±SEM. A two-tailed unpaired Welch's t-test was used to calculate P values; N=3-5 biological replicates with EVs isolated from 3-5 HBEC donors; ns=not significant.

FIG. 5 shows that Let-7b-5p reduces P. aeruginosa biofilm formation. (A) Biofilm formation by PA14-vector and PA14-let7b strains was measured in the presence of 100 mM arabinose and 300 μg/ml carbenicillin using the crystal violet 96 well plate biofilm assay (OD550). Arabinose was used to induce let-7b-5p expression and carbenicillin was used to inhibit growth of P. aeruginosa not containing the carbenicillin resistant plasmid. (B) let-7b-5p did not significantly reduce planktonic growth in biofilm plates (OD600) in the presence of 100 mM arabinose and 300 μg/ml carbenicillin and led to a significant increase in the planktonic fraction. Horizontal lines indicate means±SEM. A two-tailed unpaired Welch's t-test was used to calculate P values; N=5 replicates of independent cultures of PA14-vector or PA14-let7b strains.

FIG. 6 shows that HBEC EV (open triangles) reduced biofilm formation by 83% compared to P. aeruginosa exposed to PBS control (filled triangles). EV containing the negative control antagomir (NC EV, open diamonds) also reduced biofilm formation by 84% compared to P. aeruginosa exposed to PBS alone. By contrast, EV containing the let-7b-5p antagomir (anti-let-7b EV, filled diamonds) did not significantly reduce biofilm formation. Thus, let-7b-5p in combination with aztreonam dramatically reduces biofilm formation. Lines connect experiments conducted with HBEC and EVs from the same donor in this paired experiment. Linear mixed-effects models with HBEC donor as a random effect were used to calculate P values; N=4 biological replicates with HBEC and EVs from 4 donors; ns=not significant.

FIG. 7 shows that EV increase the ability of β-lactams to reduce P. aeruginosa biofilm formation. EV had no significant effect on planktonic P. aeruginosa in the presence of ATM (A) and CAR (B). EV inhibited biofilm formation in the presence of ATM (0.1 μg/ml) (C) and CAR (5 μg/ml) (D). EVs were isolated from six HBEC donors. **p<0.01, *p<0.05.

FIG. 8 shows that EVs isolated by two different methods reduced P. aeruginosa planktonic growth in the presence of ATM (1 μg/ml). EQ=ExoQuick-TC, OPTI=OptiPrep gradient ultracentrifugation. Data presented as means±SEM. **p<0.01 vs. ctrl.

FIG. 9 shows that EV increase the ability of β-lactams to reduce P. aeruginosa biofilm formation. EV had no significant effect on planktonic P. aeruginosa in the presence of ATM (A) and CAR (B). EV inhibited biofilm formation in the presence of ATM (0.1 μg/ml) (C) and CAR (5 μg/ml) (D). EVs were isolated from six HBEC donors. **p<0.01, *p<0.05.FIG. 10 shows that EV increase the ability of β-lactams to reduce biofilm formation by clinical isolates of P. aeruginosa. EV had no significant effect on planktonic growth in the presence of 20 μg/ml CAR (A). EV significantly reduced biofilm formation in the presence of 20 μg/ml CAR by 4 of 6 clinical isolates of P. aeruginosa (B). EV were isolated from three HBEC donors.

FIG. 10 shows that EV increases the ability of β-lactams to reduce biofilm formation by clinical isolates of P. aeruginosa. EV had no significant effect on planktonic growth in the presence of 20 μg/ml CAR (A). EV significantly reduced biofilm formation in the presence of 20 μg/ml CAR by 4 of 6 clinical isolates of P. aeruginosa (B). EV were isolated from three HBEC donors.

FIG. 11 shows that EV repress aztreonam-induced proteins. 16 P. aeruginosa

proteins (listed on the y-axis) were significantly induced by 0.1 μg/ml aztreonam (ATM) compared to controls (Ctrl, left panel). Log 2 fold changes for the comparisons are shown on the x-axis. Proteins with P<0.05 are depicted as filled red circles, while proteins with P>0.05 are shown as filled black circles. Compared to controls, EV significantly repressed 10 of the aztreonam-induced proteins (middle panel). When comparing protein levels of P. aeruginosa exposed to EV plus aztreonam to P. aeruginosa exposed to aztreonam only (right panel), all but three aztreonam-induced proteins showed lower abundance in the presence of EVs, with nine proteins showing significant repression (P<0.05). Linear models in R were used to calculate P values; N=4 biological replicates with EVs isolated from 4 HBEC donors.

FIG. 12 shows that Let-7b-5p and EV systematically repress proteins associated with biofilm formation. 29 proteins from the KEGG pathway “Biofilm Formation” that were differentially expressed in the presence of let-7b-5p compared to the empty vector control (right panel) are listed on the y-axis. Protein level changes with EV+aztreonam (ATM) compared to aztreonam alone (middle panel) and EV compared to controls in the absence of antibiotics (left panel) are shown for comparison. Log 2 fold changes for the different comparisons are shown on the x-axis. Proteins with P<0.05 are depicted as filled red circles, while proteins with P>0.05 are shown as filled black circles. Compared to the empty vector control, let-7b-5p expression significantly repressed 24 of these 29 proteins on the biofilm formation pathway (right panel). Seven of these let-7b-5p repressed proteins (Hcp3, IcmF1, PpkA, ClpV2, ClpV3, Tssk1 and HsiB1) were also significantly repressed by EV in the presence and absence of aztreonam. Linear models in R were used to calculate P values; N=4 biological replicates with EVs isolated from 4 HBEC donors (left and middle panel) or N=3 replicates of independent cultures of the empty vector or let-7b expressing strains grown in the presence of 150 μg/ml carbenicillin.

FIG. 13 shows that human bone marrow derived mesenchymal stromal cell (MSC) EV reduced Colony Forming Units (CFU) counts in Pseudomonas biofilms grown on WT-HBEC.

FIG. 14 shows that MSC EV block the formation of biofilms on primary HBEC.

FIG. 15 shows that WT (wild type) and CF HBEC EV reduce Pseudomonas in a CF mouse model of lung infection and prevents weight loss.

FIG. 16. shows that mesenchymal stromal cell (MSC) EV expressing let-7b reduce Pseudomonas in a CF mouse model of lung infection.

FIG. 17. shows that MSC EV reduced inflammation in CF mouse lung.

FIG. 18 shows the predicted targets of let-7b in other lung pathogens. All black dots below the red dotted line indicate genes that have an energy score that is significantly low to predict interaction of let-7b. P values indicate that let-7b has predicted gene targets in Burkholderia and Streptococcus as well as Pseudomonas.

### DETAILED DESCRIPTION

This disclosure provides a composition in the form of particles loaded with one or more MicroRNA (miRNA), and one or more antibiotic and methods of using such composition for treating infectious diseases.

### Definitions

Certain terms used in the specification, examples and appended claims are defined here. These definitions should be read in light of the remainder of the disclosure and understood as by a person of skill in the art. Unless defined otherwise, all technical and scientific terms used herein have the same meaning as commonly understood by a person of ordinary skill in the art. The terms used throughout this specification are defined as follows, unless otherwise limited in specific instances.

The articles “a,” “an” and “the” are used to refer to one or more than one (i.e., to at least one) of the grammatical object of the article.

The terms “comprise”, “comprising”, “including” “containing”, “characterized by”, and grammatical equivalents thereof are used in the inclusive, open sense, meaning that additional elements are not expressly mentioned but may be included. It is not intended to be construed as “consists of only.”

As used herein, the term “nanoparticle” refers to particles in the range between 10 nm to 1000 nm in diameter, wherein diameter refers to the diameter of a perfect sphere having the same volume as the particle. The term “nanoparticle” is used interchangeably as “nanoparticle(s)”. In some cases, the diameter of the particle is in the range of about 1-1000 nm, 10-500 nm, 20-300 nm, or 100-300 nm. In various embodiments, the diameter is about 35 30-170 nm. In some embodiments, the diameter of the nanoparticle is 1, 5, 10, 25, 50, 75, 100, 125, 150, 175, 200, 225, 250, 275, 300, 325, 350, 375,400, 425, 450, 475, 500, 525, 550, 575, 600, 625, 650, 675, 700, 725, 750, 775, 800, 825, 850, 875, 900, 925, 950, 975, or 1000 nm.

In some cases, a population of particles may be present. As used herein, the diameter of the nanoparticles is an average of a distribution in a particular population. The term “pharmaceutically acceptable” as used herein refers to those compounds, materials, compositions and/or dosage forms, which are, within the scope of sound medical judgment, suitable for contact with the tissues a warm-blooded animal, e.g., a mammal or human, without excessive toxicity, irritation allergic response and other problem complications commensurate with a reasonable benefit/risk ratio.

A “therapeutically effective amount” of a composition is an amount sufficient to provide an observable or clinically significant improvement over the baseline clinically observable signs and symptoms of the disorders treated with the combination.

As used herein, the term “carrier” refers to a diluent, adjuvant, excipient, or vehicle with which a compound is administered. Non-limiting examples of such pharmaceutical carriers include liquids, such as water and oils, including those of petroleum, animal, vegetable or synthetic origin, such as peanut oil, soybean oil, mineral oil, sesame oil and the like. The pharmaceutical carriers may also be saline, gum acacia, gelatin, starch paste, talc, keratin, colloidal silica, urea, and the like. In addition, auxiliary, stabilizing, thickening, lubricating and coloring agents may be used. Other examples of suitable pharmaceutical carriers are described in Remington: The Science and Practice of Pharmacy, 21^{st} Edition (University of the Sciences in Philadelphia, ed., Lippincott Williams & Wilkins 2005); and Handbook of Pharmaceutical Excipients, 7^{th} Edition (Raymond Rowe et al., ed., Pharmaceutical Press 2012); each hereby incorporated by reference in its entirety.

The term “subject” or “patient” as used herein is intended to include animals, which are suffering from or may suffer from in the near future a disease or a disorder. Examples of subjects include but are not limited to mammals, e.g., humans, apes, monkeys, dogs, cows, horses, pigs, sheep, goats, cats, mice, rabbits, rats, and transgenic non-human animals. In an embodiment, the subject is a human, e.g., a human suffering from, at risk of suffering from, or potentially capable of suffering from a disease or disorder.

The term “treating” or “treatment” as used herein comprises a treatment relieving, reducing or alleviating at least one symptom in a subject or producing a delay in the progression of a disease. For example, treatment can be the diminishment of one or several symptoms of a disorder or complete eradication of a disorder, such as cancer. Within the meaning of the present disclosure, the term “treat” also denotes to arrest and/or reduce the risk of worsening a disease. The term “prevent”, “preventing” or “prevention” as used herein comprises the prevention of at least one symptom associated with or caused by the state, disease or disorder being prevented.

As used herein, the term “extracellular vesicle” or “EV” refers to a cell-derived vesicle comprising a membrane that encloses an internal space. Extracellular vesicles include all membrane-bound vesicles (e.g., exosomes, nanovesicles) that have a smaller diameter than the cell from which they are derived. Generally extracellular vesicles range in diameter from 20 nm to 1000 nm, and can comprise various macromolecular payload either within the internal space (i.e., lumen), displayed on the external surface of the extracellular vesicle, and/or spanning the membrane. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, prokaryotic or eukaryotic cells, and/or cultured cells. In one embodiment, the extracellular vesicles are derived from a mesenchymal stem cell (MSC) or from a human epithelial cell (e.g., HBEC). In another embodiment, the extracellular vesicles are derived from a mesenchymal stem cell isolated from the same subject being treated for the infection. In another embodiment, the extracellular vesicles are derived from a primary cell culture. In another embodiment, the extracellular vesicles are not derived from a primary cell culture. In another embodiment, the extracellular vesicles are derived from a cell line (i.e., cells that are maintained and perpetuated in a lab).

As used herein the term “exosome” refers to a cell-derived small (between 20-300 nm in diameter, more preferably 40-200 nm in diameter) vesicle including a membrane that encloses an internal space (i.e., lumen), and which is generated from said cell by direct plasma membrane budding or by fusion of the late endosome with the plasma membrane. The exosome is a species of extracellular vesicle. The exosome comprises lipid or fatty acid and polypeptide and optionally comprises a payload (e.g., a therapeutic agent), a receiver (e.g., a targeting moiety), a polynucleotide (e.g., a nucleic acid, RNA, or DNA), a sugar (e.g., a simple sugar polysaccharide, or glycan) or other molecules. In some embodiments, an exosome comprises a scaffold moiety. The exosome can be derived from a producer cell, and isolated from the producer cell based on its size, density, biochemical parameters, or a combination thereof.

As used herein interchangeably, the term “microRNA,” or “miRNA” refers to the unprocessed or processed RNA transcript from a miRNA gene. MicroRNAs (miRNAs) are non-coding RNAs (typically 19-25 nucleotides in length) that regulate gene expression by inducing translational inhibition or cleavage of their target mRNA through base pairing to partially or fully complementary sites.

As used herein the term “biofilm” refers to a structured community of microorganisms enclosed in a self-produced extracellular polymeric matrix, and attached to a biotic or abiotic surface. Bacteria in a biofilm can be 1000 times more resistant to antibiotics compared to their planktonic (free living) counterparts.

As used herein, the term “biofilm formation” refers to the attachment of microorganisms to surfaces and the subsequent development of multiple layers of cells.

In one aspect, the present disclosure provides a composition comprising a) a particle selected from the group consisting of liposome, extracellular vesicle, solid lipid nanoparticles, and polymeric nanoparticles; b) a miRNA; and c) an antibiotic, wherein the particle is loaded with at least one of the miRNA and the antibiotic. In one embodiment, the particle is loaded with the miRNA. In another embodiment, the particle is loaded with the antibiotic. In another embodiment, the particle is loaded with both the miRNA and the antibiotic. In another embodiment, some particles are loaded with the miRNA, and some other particles are loaded with the antibiotic. In another embodiment, the miRNA and/or the antibiotic is encapsulated within the same particle. In another embodiment, the particles are made of materials that are non-antigenic. In another embodiment, the particles are made of materials that are non-allergenic.

In another embodiment, the particle is an extracellular vesicle (EV). The vesicles may be isolated from a eukaryotic or a prokaryotic cell. Examples of eukaryotic cells are a mammalian cell, a yeast cell or a plant cell. An example of a prokaryotic cell is a bacterial cell. In another embodiment, the extracellular vesicle is derived from an adult stem cell. In another embodiment, the extracellular vesicle is derived from a mesenchymal stem cell.

In another embodiment, the particle is a liposome. Liposomes typically include various types of lipids, phospholipids, and/or surfactants. The components of liposomes are arranged in a bilayer configuration, similar to the lipid arrangement of biological membranes (cell or EV membranes). Methods for preparation of liposomes are known in the art, for example, as provided by Epstein et al, 1985, Proc. Natl. Acad. Set USA, 82:3688; Hwang et al, 1980, Proc. Natl. Acad. Sci. USA, 77:4030-4; and U.S. Pat. Nos. 4,485,045 and 4,544,545. In addition, vesicle forming lipids can be used to formulate liposomes. Such lipids typically include two hydrocarbon chains, such as acyl chains and a polar head group.

Examples of vesicle forming lipids include phospholipids, e.g., phosphatidylcholine, phosphatidylethanolamine, phosphatidic acid, phosphatidylinositol, sphingomyelin and glycolipids, e.g., cerebrosides, gangliosides. In some embodiments, the liposomes or liposomal compositions further comprise a hydrophilic polymer, e.g., polyethylene glycol and ganglioside GM1, which increases the serum half-life of the liposome.

In another embodiment, the particle is a polymeric nanoparticle. In another embodiment, the particle is a biodegradable polymeric nanoparticle. In another embodiment, the particle is a biocompatible polymeric nanoparticle. In one embodiment, the polymeric nanoparticle is made from polymers. Examples of biodegradable polymers include polymers of hydroxy acids such as lactic acid and glycolic acid, and copolymers with PEG, polyanhydrides, poly(ortho)esters, polyurethanes, poly(hydroxy butyric acid), poly(hydroxy valeric acid), poly(lactide-co-caprolactone), poly(amine-co-ester), blends and copolymers thereof. In some embodiments, the particles are composed of one or more polyesters.

In some embodiments, the one or more polyesters are hydrophobic. For example, particles can contain one more of the following polyesters: homopolymers including glycolic acid units (PGA), and lactic acid units, such as poly-L-lactic acid, poly-D-lactic acid, poly-D,L-lactic acid, poly-L-lactide, poly-D-lactide, and poly-D,L-lactide, and caprolactone units, such as poly(s-caprolactone); and copolymers including lactic acid and glycolic acid units, such as various forms of poly(lactic acid-co-glycolic acid) and poly(lactide-co-glycolide) characterized by the ratio of lactic acid: glycolic acid; and polyacrylates, and derivatives thereof.

Suitable hydrophilic polymers include, but are not limited to, hydrophilic polypeptides, such as poly-L-glutamic acid, gamma-polyglutamic acid, poly-L-aspartic acid, poly-L-serine, or poly-L-lysine, poly(alkylene glycols) such as polyethylene glycol (PEG), poly(propylene glycol) and copolymers of ethylene glycol and propylene glycol, poly(oxyethylated polyol), poly(olefinic alcohol), polyvinylpyrrolidone), poly(hydroxyalkylmethacrylamide), poly(hydroxyalkylmethacrylate), poly(saccharides), poly (hydroxyacids), poly(vinyl alcohol), as well as copolymers thereof. In some embodiments, the hydrophilic polymer is PEG.

In some embodiments, the polymers are amphiphilic containing a hydrophilic and a hydrophobic polymer. Exemplary amphiphilic polymers also include copolymers of PEG and polyesters, such as various forms of PLGA-PEG or PLA-PEG copolymers. In some embodiments, the PEG region can be covalently associated with polymer to yield “PEGylated polymers” by a cleavable linker.

In another embodiment, the particle is a solid lipid nanoparticle. Solid lipid nanoparticles are formed of lipids that are solids at room temperature. In one embodiment, the solid lipid nanoparticles have an average diameter between 10 and 1000 nanometers. Suitable solid lipids include, but are not limited to, higher saturated alcohols, higher fatty acids, sphingolipids, synthetic esters, and mono-, di-, and triglycerides of higher saturated fatty acids. Solid lipids can include aliphatic alcohols having 10-40, preferably 12-30 carbon atoms, such as cetostearyl alcohol. Solid lipids can include higher fatty acids of 10-40, preferably 12-30 carbon atoms, such as stearic acid, palmitic acid, decanoic acid, and behenic acid. Solid lipids can include glycerides, including monoglycerides, diglycerides, and triglycerides, of higher saturated fatty acids having 10-40 or 12-30 carbon atoms, such as glyceryl monostearate, glycerol behenate, glycerol palmitostearate, glyceryl trilaurate, tricaprin, trilaurin, trimyristin, tripalmitin, tristearin, and hydrogenated castor oil. Suitable solid lipids can include cetyl palmitate, beeswax, or cyclodextrin.

In another embodiment, the antibiotic is selected from the group consisting of β-lactam antibiotics, fluoroquinolone antibiotics, and aminoglycoside antibiotics. In another embodiment, the antibiotic is selected from the group consisting of aztreonam, tobramycin, carbenicillin, azithromycin, colistin/polymyxin E, gentamicin and ciprofloxacin. In one embodiment, the amount of each of these antibiotics loaded onto the EV or NP is an amount of the antibiotic that, in conjunction with the microRNA (e.g., let-7b), is effective in inhibiting proliferation of Pseudomonas aeruginosa in the infected subject. In another embodiment, the amount of each of these antibiotics loaded onto the EV or NP is in the range of from about 1 μg to about 1 g per vesicle/particle. In another embodiment, the amount of each of these antibiotics loaded onto the EV or NP is in the range of from about 1 μg to about 1 mg per vesicle/particle. In another embodiment, the amount of each of these antibiotics loaded onto the EV or NP is in the range of from about 1 mg to about 100 mg per vesicle/particle.

In another embodiment, the composition is an inhalable powder, an aerosol, or a spray. In another embodiment, the composition is adapted for aerosolized administration. In another embodiment, the particle has a diameter ranging from 10 nm to 1,000 nm.

In one aspect, the concentration of aztreonam to be loaded onto the vesicle/particle is the amount, when inhaled, reaches between 100 μg/ml and 1000 μg/ml, or about 700 20 μg/ml in the sputum of the subject. In another aspect, the concentration of tobramycin to be loaded onto the vesicle/particle is the amount, when inhaled, reaches between 1000 μg/ml and 2000 μg/ml, or about 1200 μg/ml in the sputum of the subject. In another aspect, the concentration of carbenicillin to be loaded onto the vesicle/particle is the amount, when inhaled, reaches between 1000 μg/ml and 2000 μg/ml, or about 1200 μg/ml in the sputum of the subject. In another aspect, the concentration of ciprofloxacin to be loaded onto the vesicle/particle is the amount, when inhaled, reaches between 30 μg/ml and 300 μg/ml, or between 50 μg/ml and 200 μg/ml in the sputum of the subject. In another aspect, the concentration of azithromycin to be loaded onto the vesicle/particle is the amount, when inhaled, reaches between 5 μg/ml and 100 μg/ml, or between 10 μg/ml and 20 μg/ml in the sputum of the subject. In another aspect, the concentration of colistin/polymyxin E to be loaded onto the vesicle/particle is the amount, when inhaled, reaches between 10 μg/ml and 100 μg/ml, or about 40 μg/ml in the sputum of the subject. In another aspect, the concentration of gentamicin to be loaded onto the vesicle/particle is the amount, when inhaled, reaches between 50 μg/ml and 200 μg/ml, or between 80 μg/ml and 120 μg/ml in the sputum of the subject.

In another aspect, the present disclosure provides a pharmaceutical composition including the composition disclosed herein and a pharmaceutically acceptable carrier.

In another aspect, the present disclosure provides a kit including the composition disclosed herein and instructions for use.

In another aspect, the present disclosure provides a method of inhibiting proliferation of biofilm-embedded microorganisms including administering a therapeutically effective amount of the composition disclosed herein.

In another aspect, the present disclosure provides a method of preventing or treating a P. aeruginosa infection in a subject in need thereof, including administering to the subject a therapeutically effective amount of the composition disclosed herein.

In another aspect, the present disclosure provides a method of reducing proliferation, survival, migration, or colony formation ability of a rapidly proliferating cell in a subject in need thereof including contacting the cell with a therapeutically effective amount of the composition disclosed herein.

In another aspect, the present disclosure provides a method of reducing proliferation, survival, migration, or colony formation ability of a microorganism in a subject in need thereof including contacting the microorganism with a therapeutically effective amount of the composition disclosed herein. In one embodiment, the microorganism is P. aeruginosa.

In one embodiment, the composition is administered in an aerosolized form. In another embodiment, the composition is administered by inhalation. In another embodiment, the composition is administered by nasal inhalation. In another embodiment, the composition may be delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount.

In another aspect, the present disclosure provides a method of preventing or treating a P. aeruginosa infection in a subject in need thereof, including administering to the subject an extracellular vesicle loaded with let-7b in combination with an antibiotic. In one embodiment, the infection is a chronic lung infection. In another embodiment, the infection is a respiratory tract infection.

In one embodiment, let-7b, a 22-nucleotide miRNA increases the ability of antibiotics to kill planktonic P. aeruginosa and to significantly reduce the ability of P. aeruginosa to form antibiotic resistant biofilms.

In one aspect, the present disclose provides extracellular vesicles loaded with a miRNA isolated, or isolated and purified, from a biological sample of a subject or from a culture medium via suitable isolation methods. Examples of isolation methods include ExoQuick-TC® (EQ) and OptiPrep™ gradient ultracentrifugation (OPTI).

In one embodiment, electroporation is used to load a particle with miRNA and antibiotics. In another embodiment, the particle is EV. In another embodiment, the particle is mesenchymal stem cells (MSC) EV.

In one aspect, the present disclosure provides a method of reducing or preventing biofilm formation by a microorganism on a living or nonliving surface comprising treating the surface with an effective amount of the composition disclosed herein. In one embodiment, the microorganism is P. aeruginosa.

### EXAMPLES

The disclosure will now be illustrated with working examples, and which is intended to illustrate the working of disclosure and not intended to restrictively any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

### Example 1 Extracellular Vesicles Secreted by Human Bronchial Epithelial Cells Enhance Drug Delivery

This example shows that extracellular vesicles (EVs) secreted by primary cultures of human bronchial epithelial cells (HBEC) containing let-7b deliver let-7b to P. aeruginosa.

Analysis of the RNA content in EV, and EV delivery of let-7b to P. aeruginosa. To assess the RNA content of EV secreted by HBEC, RNA-seq was conducted and the results analyzed as described^{26,27} (n=3 donors). Several classes of small RNAs were identified including tRNA, tRNA-like fragments, rRNA, piRNA, lincRNA, and miRNA, consistent with previous reports of RNA content of EVs secreted by other cell types²⁸⁻³⁴. The five most abundant miRNAs in EV secreted by HBEC, miR-320a, let-7b-5p, let-7a-5p, miR-26a, and miR-1246, accounted for over 50% of all mature miRNA sequence reads.

To determine if EV deliver miRNAs and let-7b to P. aeruginosa, RNA-seq experiments of P. aeruginosa exposed to EV and unexposed controls were conducted. P. aeruginosa was exposed to vehicle control or EV at a ratio of 1000 EV per bacterium and corresponding to a concentration of EVs measured in HBEC supernatants by Nanoparticle tracking analysis (NTA) (n=3 donors). To avoid possible carry-over of EVs (and miRNA) attached to the outside of the bacteria, samples were washed with PBS and the bacterial associated with the bacterial outer membrane and periplasm³⁵⁻³⁷. The cells were then washed again in PBS, and RNA was harvested after bacterial lysis. Total RNA was isolated by miRNeasy including the on-column DNA digestion. Sequencing libraries were prepared with the QIAseq smRNA kit and 75 bp single-end reads were generated using an Illumina MiniSeq. Sequences that did not map to the PA14 reference genome were aligned to the human genome using CLC Genomics Workbench software. Six mature human miRNAs from the let-7 family in P. aeruginosa (strain PA14) that had been exposed to EVs (let-7a-5p, let-7b-5p, let-7f-5p, let-7e-5p, let-7c-5p and let-7g-5p) were detected.

EVs secreted by eukaryotic cells deliver miRNA into the cytoplasm of a prokaryote. The miRNAs transferred to P. aeruginosa by EVs might regulate P. aeruginosa gene expression by targeting bacterial mRNAs. Let-7b targets all three subunits of an Resistance-Nodulation-Division (RND) efflux pump (mexGHI-opmD) for the efflux of fluoroquinolone antibiotics^{38, 39}, several β-lactamases that degrade β-lactam antibiotics (PA14_72760, PA14_26350, PA14_00690, and PA14_175704⁴⁰, as well as nirS, pchA, pchE, pchF, and pchG, which play key roles in the development of antibiotic resistant biofilms. Let-7b increases the ability of anti-pseudomonal antibiotics like ciprofloxacin, carbenicillin and aztreonam, which are used in the clinic to treat P. aeruginosa infections, to kill P. aeruginosa and inhibit biofilm formation. Table 1 shows list of proteins repressed by let-7b-5p.

TABLE 1
Proteins repressed by let−7b−5p

| Locus | Name | Product | log2 FC | p-value | KEGG Pathway | Energy |
|---|---|---|---|---|---|---|
| PA14_00310 | PA14_00310 | peptidyl-prolyl isomerase | −0.7 | 0.0314 | | −16.1 |
| PA14_00875 | PpkA | serine/threonine protein Kinase | −0.6 | 0.0172 | Biofilm formation; Bacterial secretion system | −19.2 |
| PA14_00890 | PppA | phosphoprotein phosphatase | −1.1 | 0.0045 | Biofilm formation; Bacterial secretion system | −15.4 |
| PA14_00940 | TsaK1 | hypothetical protein | −0.9 | 0.0138 | Biofilm formation | −15.4 |
| PA14_00960 | TssJ1 | lipoprotein | −0.9 | 0.0328 | Bacterial secretion system | −15.3 |
| PA14_01010 | HsiB1 | hypothetical protein | −1.5 | 0.0118 | Biofilm formation | −13.1 |
| PA14_01020 | HsiC1 | hypothetical protein | −1.5 | 0.0037 | Biofilm formation | −17.5 |
| PA14_01030 | Hcp1 | hypothetical protein | −1.6 | 0.0210 | Biofilm formation; Bacterial secretion system | −11.0 |
| PA14_01100 | ClpV1 | ClpA/B-type chaperone | −1.5 | 0.0128 | Biofilm formation; Bacterial secretion system | −17.7 |
| PA14_01110 | VgrG1a | hypothetical protein | −0.7 | 0.0403 | Bacterial secretion system | −13.8 |
| PA14_01150 | EagT6 | hypothetical protein | −0.9 | 0.0088 | | −14.1 |
| PA14_02180 | CheB | chemotaxis-specific methylesterase | −0.6 | 0.0218 | Two-component system; Bacterial chemotaxis | −15.3 |
| PA14_02190 | PA14_02190 | hypothetical protein | −0.7 | 0.0108 | Bacterial chemotaxis | −15.9 |
| PA14_02220 | PA14_02220 | chemotaxis transducer | −1.0 | 0.0074 | Two-component system; Bacterial chemotaxis | −11.6 |
| PA14_02230 | CheW | purine-binding chemotaxis protein | −1.0 | 0.0080 | Two-component system; Bacterial chemotaxis | −11.0 |
| PA14_02250 | CheA | two-component sensor | −1.0 | 0.0078 | Two-component system; Bacterial chemotaxis | −16.1 |
| PA14_02260 | PA14_02260 | two-component response regulator | −0.9 | 0.0094 | Two-component system; Bacterial chemotaxis | −15.0 |
| PA14_02500 | ExbB1 | transport protein | −0.7 | 0.0123 | | −15.6 |
| PA14_03610 | PA14_03610 | Zn-dependent protease/chaperone | −1.1 | 0.0343 | | −13.5 |
| PA14_04290 | PA14 04290 | hypothetical protein | −0.7 | 0.0146 | | −20.2 |
| PA14_06080 | CreD | hypothetical protein | −0.8 | 0.0083 | | −20.1 |
| PA14_09400 | PhzS | hypothetical protein | −0.8 | 0.0372 | Phenazine biosynthesis | −16.8 |
| PA14_09490 | PhzM | phenazine-specific methyltransferase | −1.3 | 0.0327 | Phenazine biosynthesis | −14.2 |
| PA14_16200 | PA14_16200 | hypothetical protein | −0.6 | 0.0231 | | −14.5 |
| PA14_16330 | PA14_16330 | hypothetical protein | −0.6 | 0.0216 | | −11.9 |
| PA14_19700 | PA14_19700 | aldolase | −0.7 | 0.0115 | | −19.2 |
| PA14_21020 | PA14_21020 | non-ribosomal peptide synthetase | −0.7 | 0.0051 | | −22.7 |
| PA14_23460 | OrfN | group 4 glycosyl transferase | −3.6 | 0.0001 | Peptidoglycan biosynthesis | −12.2 |
| PA14_24170 | FadH1 | 2,4-dienoyl-CoA reductase | −0.6 | 0.0329 | | −14.6 |
| PA14_26280 | PA14_26280 | chemotaxis transducer | −0.7 | 0.0129 | Two-component system; Bacterial chemotaxis | −16.1 |
| PA13_33990 | ClpV3 | ClpV3 | −0.8 | 0.0039 | Biofilm formation; Bacterial secretion system | −17.7 |
| PA14_36530 | PA14_36530 | hypothetical protein | −0.6 | 0.0430 | | −15.4 |
| PA14_37880 | SppA | ABC transporter substrate-binding | −0.7 | 0.0191 | ABC transporters | −13.4 |
| PA14_39780 | PA14_39780 | hypothetical transport protein | −1.1 | 0.0316 | | −14.6 |
| PA14_40110 | PA14_40110 | hypothetical protein | −0.6 | 0.0254 | | −18.2 |
| PA14_41420 | PA14_41420 | hypothetical protein | −1.4 | 0.0063 | | −13.2 |
| PA14_42980 | ClpV2 | ClpV2 | −0.7 | 0.0121 | Biofilm formation; Bacterial secretion system | −19.7 |
| PA14_45130 | PA14_45130 | transporter | −1.6 | 0.0191 | | −15.8 |
| PA14_46750 | PA14 46750 | hypothetical protein | −0.7 | 0.0477 | | −18.5 |
| PA14_46760 | PA14_46760 | hypothetical protein | −0.6 | 0.0405 | | −10.1 |
| PA14_50570 | PA14_50570 | hypothetical protein | −0.7 | 0.0102 | | −17.9 |
| PA14_50610 | PA14_50610 | short chain dehydrogenase | −0.6 | 0.0325 | | −16.1 |
| PA14_55890 | PA14_55890 | type II secretion system protein | −0.8 | 0.0245 | Bacterial secretion system | −16.3 |
| PA14_64520 | PA14_64520 | bacterioferritin | −1.5 | 0.0498 | Porphyrin and chlorophyll metabolism | −13.8 |
| PA14_70690 | GlcD | glycolate oxidase subunit | −0.8 | 0.0396 | Glyoxylate and dicarboxylate metabolism; | −12.9 |
| | | | | | Biosynthesis of antibiotics | |
| PA14_70940 | BetA | choline dehydrogenase | −0.7 | 0.0079 | Glycine, serine and threonine metabolism | −13.4 |
| PA14_71240 | PA14_71240 | hypothetical protein | −0.8 | 0.0276 | | −12.8 |
| PA14_71420 | PA14_71420 | ferredoxin | −0.7 | 0.0063 | | −14.6 |

### Example 2. EV Increase P. aeruginosa Sensitivity to Fluoroquinolone Antibiotics by Reducing the RND Efflux Pump MexGHI-OpmD

This example shows that EV containing let-7b enhances the ability of fluoroquinolone antibiotics to kill P. aeruginosa. EV targets MexGHI-OpmD, a multidrug efflux pump that contributes to resistance to fluoroquinolone antibiotics including ciprofloxacin. Hyper-expression of RND multidrug efflux pumps is frequent in clinical isolates of P. aeruginosa and contributes to the development of multi-drug resistance phenotypes in the clinical setting¹⁷. To determine if EV containing let-7b target and reduce MexGHI-OpmD, an unbiased proteomic (LC-MS/MS) approach was used, as previously described 27. EV exposure significantly (P<0.05) reduced the abundance of MexI (50%), MexH (48%) and OpmD (35%) in P. aeruginosa.

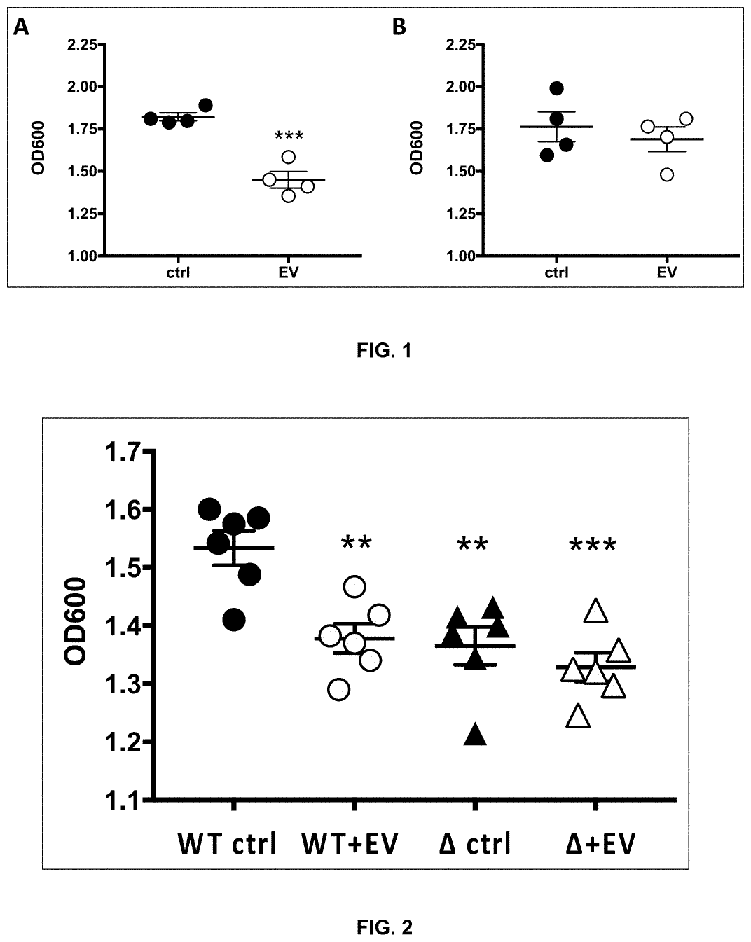

To test whether the reduction in MexGHI-OpmD increased the fluoroquinolone sensitivity of P. aeruginosa, the effect of EV on the planktonic growth of P. aeruginosa (PA14) was measured in the absence or presence of ciprofloxacin (CIP, 0.015 μg/ml), a dose corresponding to half of the minimum inhibitory concentration (MIC: the lowest concentration of antibiotic that prevents growth) for this strain. EV from HBEC were used at a concentration of 5×10⁹/ml, the EV concentration in HBEC culture supernatants as well as in BALF (5.9×10⁹/ml for healthy BALF and 2×10⁹/ml for CF-BALF, P<0.05). EV increased the CIP sensitivity of P. aeruginosa (FIG. 1A), as measured by optical density (OD600), a standard way to measure planktonic growth of bacteria⁴¹⁻⁵⁹. EV had no effect on planktonic growth in the absence of CIP (FIG. 1B).

To determine if EV increased P. aeruginosa fluoroquinolone sensitivity by directly targeting the MexGHI-OpmD efflux pump, the CIP sensitivity of a mexGHI-opmD deletion mutant in strain PAO1 and its matched parental wild type strain were assessed³⁹. If EV increased P. aeruginosa fluoroquinolone sensitivity by targeting MexGHI-OpmD, a mexGHI-opmD deletion strain of P. aeruginosa would be predicted to show increased sensitivity to CIP even in the absence of EV and exposure to EV should have no additional effect. Experiments presented in FIG. 2 were conducted in the presence of CIP (0.03 μg/ml, one-half the MIC for PAO1). EV decreased planktonic growth of the parental strain (WT ctrl versus WT+EV). Deletion of mexGHI-opmD (Δ ctrl) reduced planktonic growth (Δ ctrl compared to WT ctrl). Exposure to EV had no additional effect on planktonic growth of the knockout strain of PAO1 (compare Δ ctrl and Δ ctrl+EV).

These data are consistent with the conclusion that EV increase P. aeruginosa sensitivity to CIP by reducing the RND efflux pump MexGHI-OpmD. The fact that neither EV alone nor the mexGHI-opmD deletion strain led to a complete inhibition of P. aeruginosa growth in the presence of CIP can be explained by the expression of additional fluoroquinolone efflux pumps, such as MexEF-OprN or MexPQ-OpmE, in P. aeruginosa. EV containing let-7b decreases the antibiotic resistance of P. aeruginosa by down regulating mexGHI-opmD.

### Example 3. EV Containing let-7b Inhibits the Formation of P. aeruginosa Biofilms by Targeting Genes Essential for Biofilm Formation

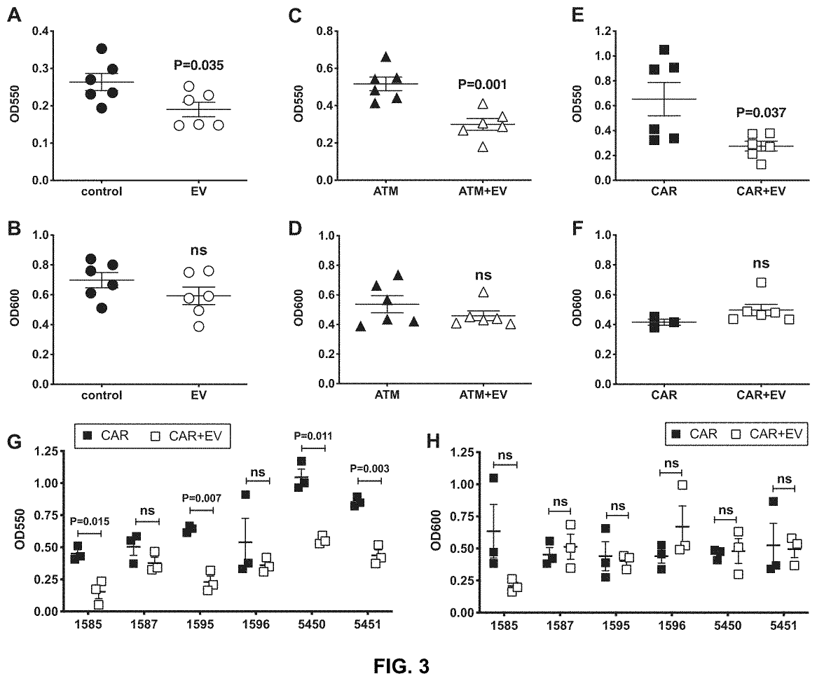

Let-7b targets several biofilm genes in P. aeruginosa, an effect that may reduce biofilm formation. Proteomic analysis of P. aeruginosa revealed that EV significantly (P<0.05) reduced protein abundance of eleven genes involved in biofilm formation (Table 1). LC-MS/MS data support that let-7b targets biofilm genes (Table 1). For example, NirS is essential for biofilm formation, and a NirS transposon mutant is deficient in biofilm formation¹⁴. NirS is a nitrite reductase that catalyzes the reduction of NO₂ to NO. While NO mediates disruption of biofilms, it is also required for initial biofilm formation, so a reduction in NO through reduced levels of NirS may interfere with biofilm formation Collectively, the EV-mediated reduction in these proteins may suppress P. aeruginosa biofilm formation. EVs reduce the ability of P. aeruginosa to form biofilms To assess whether EVs inhibit biofilm formation by P. aeruginosa, experiments were conducted using the crystal violet biofilm plate assay. The effects of EVs were examined, at a concentration observed in bronchoalveolar lavage fluid. EVs reduced biofilm formation by P. aeruginosa by 28% compared to PBS vehicle control (FIG. 3A), while they did not significantly alter planktonic growth in biofilm plates (FIG. 3B).

EVs enhance the inhibition of biofilm formation by antibiotics The crystal violet biofilm plate assay was used to determine whether EVs increase the ability of antibiotics to inhibit biofilm formation by P. aeruginosa. We examined the effect of EVs, at a concentration observed in bronchoalveolar lavage fluid, in combination with beta-lactam antibiotics, aztreonam and carbenicillin, and observed that the combination of EVs and sub-inhibitory doses of aztreonam and carbenicillin significantly reduced biofilm formation (FIG. 3C and 3E). The sub-inhibitory antibiotic concentration for biofilm formation by P. aeruginosa strain PA14 in the absence of EVs was 0.1 μg/ml for aztreonam and 5 μg/ml for carbenicillin. At these concentrations, antibiotics alone did not significantly alter biofilm formation by P. aeruginosa. In combination with EVs, aztreonam reduced biofilm formation by 42% (FIG. 3C), while planktonic growth in biofilm plates was not significantly different (FIG. 3D). Likewise, the combination of EVs and carbenicillin reduced biofilm formation by 58% (FIG. 3E), without significantly affecting planktonic growth (FIG. 3F). To determine whether these findings generalize to clinically relevant strains of P. aeruginosa, we assessed the ability of EVs to reduce biofilm formation by six clinical isolates using a concentration of carbenicillin (20 μg/ml), which did not significantly reduce biofilm formation in the absence of EVs. In combination, carbenicillin and EVs significantly decreased biofilm formation by four of the six clinical isolates we tested (FIG. 3G), demonstrating that the effect of EVs to prevent biofilm formation by P. aeruginosa is clinically relevant and not limited to the PA14 strain.

The combination of EVs and carbenicillin reduced biofilm formation by clinical Isolate 1585 by 66%, 1595 by 64%, 5450 by 47%, and 5451 by 49% (FIG. 3G). EVs did not significantly reduce planktonic growth of P. aeruginosa in biofilm plates in the presence of carbenicillin in any of the six clinical isolates (FIG. 3H). It was also assessed whether EVs increase the ability of aztreonam to inhibit biofilm formation by clinical isolates of P. aeruginosa. It was found that the combination of aztreonam and EVs significantly inhibited biofilm formation by clinical isolates 1585 and 1595. EVs in combination with a sub-inhibitory concentration of aztreonam (0.5 μg/ml) reduced biofilm formation by clinical isolate 1585 by 65%, concomitant with a 10-fold reduction in biofilm colony forming units (CFUs), while planktonic growth (OD 600) and planktonic CFUs were not significantly altered compared to aztreonam alone. Likewise, 0.5 μg/ml aztreonam in combination with EVs reduced biofilm formation by clinical isolate 1595 by 50%, and reduced biofilm CFUs 10-fold, but did not significantly affect planktonic growth or planktonic CFUs.

Studies were also conducted to examine the time course of EV inhibition of biofilm formation using the crystal violet plate assay. While no biofilms were detected after 6 hours of incubation in any condition, biofilms were detected after 12 hours and increased further after 24 hours for control as well as aztreonam and carbenicillin alone. EVs alone (control+EV), EVs+aztreonam and EVs+carbenicillin reduced biofilm formation at 12 hours and 24 hours compared to control, aztreonam alone and carbenicillin alone. Thus, EVs alone and in the presence of aztreonam and carbenicillin significantly reduced biofilm formation at 12 and 24 hours.

Table 2 shows let-7b target genes implicated in biofilm formation and changed of their abundance induced by EV. Column 1-Genes involved in biofilm formation (PhzE1 and PhzE2 have been identified as targets of let-7b in preliminary experiments). Let-7b may bind to the other biofilm genes, such as the genes listed in Table 1. Column 2-Percent change in protein abundance induced by EV, determined by LC-MS/MS. Column 3-IntaRNA targeting score for let-7b for each biofilm gene, indicating a highly energetically favorable interaction between let-7b and the target mRNA⁵¹.

TABLE 2
Let-7b target genes implicated in biofilm formation

| Biofilm Gene | Percent Change in Protein | let-7b targeting score |
|---|---|---|
| NarG | −46% | −14.2 |
| NarH | −40% | −14.3 |
| NarI | −45% | −10.7 |
| NirS | −62% | −15.7 |
| NosZ | −74% | −16.7 |
| PvdA | −60% | −15.4 |
| PvdD | −37% | −14.7 |
| PvdH | −28% | −15.5 |
| PvdJ | −35% | −18.8 |
| PvdQ | −34% | −14.3 |
| PhzE1 | −20% | −18.2 |
| PhzE2 | −21% | −18.2 |

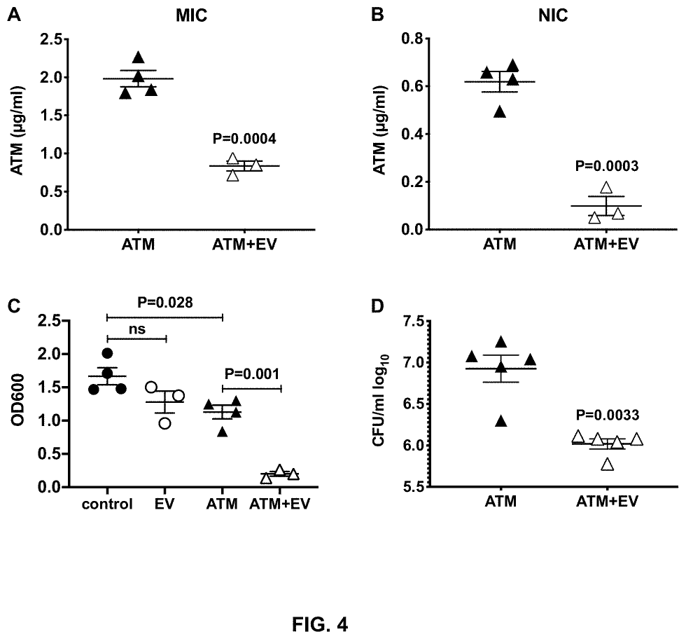

EVs increase the beta-lactam sensitivity of planktonic P. aeruginosa To determine whether EVs also affect planktonic P. aeruginosa, planktonic growth yield assays were performed over a range of 0-25 μg/ml aztreonam in the presence or absence of EVs. It was found that EVs decreased both the minimal inhibitory concentration (MIC) and non-inhibitory concentration (NIC) for aztreonam (FIG. 4). EVs decreased the MIC of aztreonam more than 2-fold in the presence of EVs (FIG. 4A). EVs also induced a 6-fold decrease in the NIC of aztreonam (FIG. 4B). Moreover, in the presence of aztreonam (0.8 μg/ml, about one-half the MIC for P. aeruginosa strain PA14), EVs significantly reduced P. aeruginosa planktonic yield (FIG. 4C) as well as CFUs (FIG. 4D), compared to P. aeruginosa not exposed to EVs. Aztreonam at a concentration close to one-half the MIC (0.8 μg/ml) significantly reduced planktonic growth compared to controls (FIG. 4C). There was a trend for EVs to reduce planktonic growth in the absence of aztreonam, but it did not reach statistical significance (FIG. 4C). Compared to aztreonam alone, the combination of aztreonam and EVs first led to a statistically significant reduction in planktonic growth at 15 hours and 15 minutes and remained significantly repressed throughout the remainder of the time course. Importantly, the finding that a combination of EVs and aztreonam reduces planktonic growth of P. aeruginosa was independent of the method used to isolate EVs. Taken together with the biofilm experiments, these findings demonstrate that eukaryotic EVs increase the beta-lactam sensitivity of planktonic P. aeruginosa and reduce the ability of P. aeruginosa to form biofilms.

To elucidate the mechanism whereby EVs reduce biofilm formation and beta-lactam antibiotic sensitivity, an RNA-seq analysis of EVs secreted by primary human HBEC was performed and several classes of small RNAs in EVs were identified, including tRNA, tRNA-like fragments, rRNA, piRNA, lincRNA, and miRNA, which is consistent with previous reports of RNA content of EVs secreted by other eukaryotic cells²⁸⁻³⁴. The five most abundant miRNAs in EVs secreted by HBEC were miR-320a, let-7b-5p, let-7a-5p, miR-26a-5p, and miR-1246, accounting for >50% of all miRNA sequence reads. To determine whether EVs can deliver miRNAs to P. aeruginosa, an RNA-seq analysis of P. aeruginosa exposed to EVs or vehicle was conducted. To avoid possible carry-over of EVs (and miRNA) attached to the outside of the bacteria, after exposure to EVs the bacterial outer membrane was lysed with EDTA prior to RNA isolation. Cytoplasmic RNA was isolated after lysis of the cell wall and inner membrane. Six mature human miRNAs were detected from the let-7 family (let-7a-5p, let-7b-5p, let-7c-5p, let-7e-5p, let-7f-5p, and let-7g-5p) in P. aeruginosa exposed to EVs, confirming the hypothesis that EVs can deliver miRNAs to the cytoplasm of P. aeruginosa. This was the first direct demonstration that EVs secreted by a eukaryotic organism deliver miRNAs to a prokaryotic organism.

miRNA targeting prediction algorithm IntaRNA 52 was used to assess whether the six miRNAs transferred to P. aeruginosa by EVs are predicted to regulate P. aeruginosa gene expression by targeting bacterial mRNAs. IntaRNA was designed to predict mRNA target sites for eukaryotic miRNAs or bacterial small RNAs based on RNA-RNA interactions due to sequence similarity.

For each potential miRNA and mRNA target pair, IntaRNA calculates a combined energy score of the interaction that includes the free energy of hybridization as well as the free energy required for making the interaction sites accessible. The lower the energy score, the higher the likelihood of a successful targeting interaction. It was found that among the six miRNA that were transferred from EVs to P. aeruginosa, let-7b-5p had by far the most predicted high-quality P. aeruginosa gene targets, including genes that play an important role in biofilm formation and antibiotic resistance. Therefore let-7b-5p was selected for follow-up experiments to test the hypothesis that let-7b-5p, delivered by EVs, increases the ability of beta-lactam antibiotics to reduce P. aeruginosa biofilm formation. To determine if the mechanism of action of P. aeruginosa targeting by let-7b-5p involves interaction of let-7b with regulatory intergenic regions such as UTRs, rather than direct interaction within coding regions of genes, IntaRNA was used to predict let-7b-5p targeting of P. aeruginosa intergenic regions. It was found that the average IntaRNA energy score for P. aeruginosa intergenic regions of −8.44 was significantly higher (P=0) and thus much worse than the average energy score for P. aeruginosa gene coding regions of −14.62. This result suggests that let-7b-5p is more likely to regulate P. aeruginosa gene expression by directly targeting P. aeruginosa genes as opposed to intergenic regulatory regions.

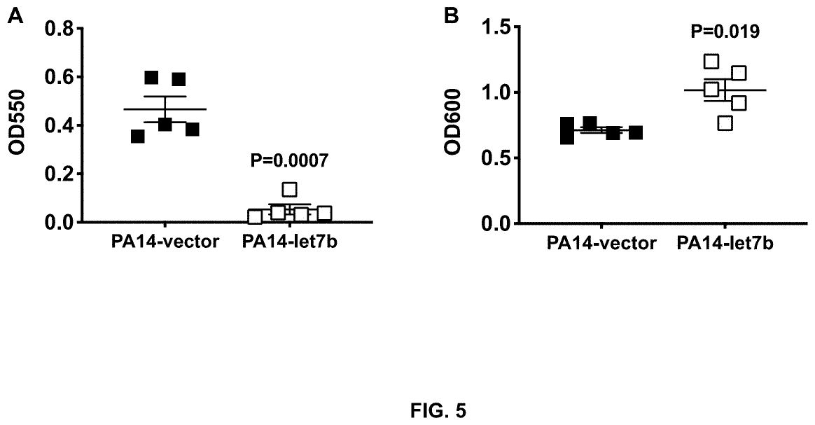

To provide direct evidence for the hypothesis that let-7b-5p reduces biofilm formation and increases the ability of beta-lactam antibiotics to reduce biofilm formation, we generated a PA14 strain (PA14-let7b) that expresses let-7b-5p under an arabinose-inducible promoter. A crystal violet biofilm plate assay was performed with PA14-let7b and a PA14 strain expressing the empty pMQ70 plasmid (PA14-vector). Biofilm formation by PA14-let7b was 90% less than biofilms formed by PA14-vector (FIG. 5A). This finding is consistent with the hypothesis that let-7b-5p decreases biofilm formation. By contrast, there was a small increase in planktonic PA14-let7b compared to PA14-vector (FIG. 5B). Recognizing that let-7b-5p concentrations in a genetically engineered strain might exceed those found in P. aeruginosa exposed to EVs, additional studies were conducted to examine the ability of a let-7b-5p antagomir to antagonize endogenous levels of let-7b-5p in EVs and block the ability of EVs to reduce biofilm formation by P. aeruginosa growing on HBEC.

As shown previously, co-culture of P. aeruginosa grown on a biotic surface such as primary HBEC, rather than an abiotic surface like the 96-well plastic plates used in the crystal violet biofilm plate assay, represents a biologically relevant model to study the formation of P. aeruginosa biofilms that is comparable to in vivo models. To test the hypothesis that let-7b-5p inhibits biotic biofilm formation, P. aeruginosa was exposed to EVs isolated from HBEC transfected with a let-7b-5p antagomir (anti-let-7b EV), EVs isolated from HBEC transfected with a miRNA negative control (NC EV), EVs isolated from untransfected HBEC, or PBS vehicle control.

In prior crystal violet biofilm and planktonic growth curve experiments we observed that it takes 12 hours and more than 15 hours, respectively, for EVs to reduce planktonic growth and biofilm formation of P. aeruginosa, presumably due to the long half-life of proteins targeted by let-7b-5p. We therefore pre-exposed P. aeruginosa to a biologically relevant concentration of EVs or PBS as a vehicle control for 18 hours before adding P. aeruginosa to the HBEC. EVs used for pre-exposures were derived from the same airway cell donor that was used in the subsequent co-culture experiment, which was performed with a total of four donors.

P. aeruginosa biofilms were imaged after 6 hours of co-culture, a time point that is too short for EVs produced by the HBEC during co-culture to affect biofilm formation, based on previous time course experiments. Because P. aeruginosa is cytotoxic to HBEC after 4-9 hours 46, it was not possible to examine the effect of EVs directly secreted by HBEC in co-culture. Moreover, even if a prolonged co-culture of P. aeruginosa and HBEC were possible, such an experimental design would not allow for a no EV control, as airway cells constitutively secrete EVs. The 18-hour pre-exposures as well as the 6-hour co-cultures included a low concentration of aztreonam (0.1 μg/ml) that by itself did not affect planktonic growth or biofilm formation of P. aeruginosa (FIG. 3D). P. aeruginosa exposed to PBS vehicle control formed robust biofilms after 6 hours (data not shown), while P. aeruginosa that had been pre-exposed to EVs for 18 h showed dramatically reduced biofilm formation (data not shown). Likewise, pre-exposure of P. aeruginosa to EVs secreted by HBEC that had been transfected with a miRNA negative control (NC EV) induced a robust reduction of biofilm formation (data not shown). By contrast, pre-exposure of P. aeruginosa to EVs harvested from HBEC transfected with the let-7b-5p antagomir (anti-let-7b EV) did not significantly reduce P. aeruginosa biofilm formation (data not shown).

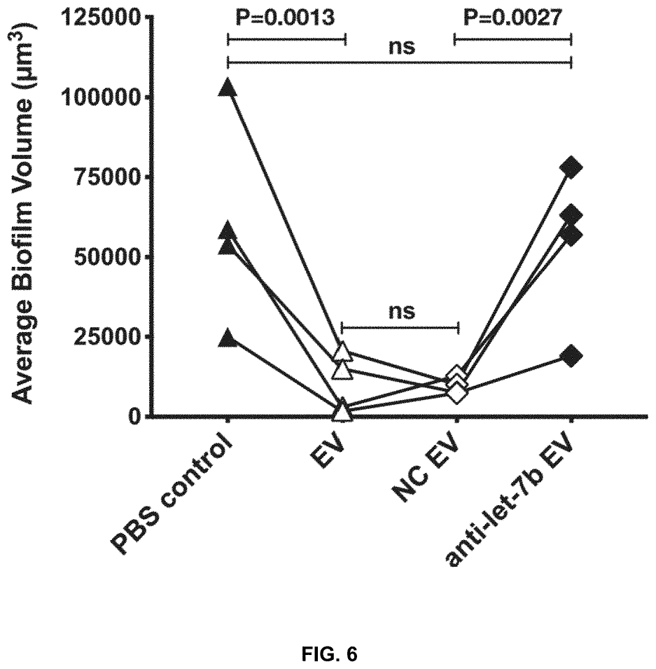

In summary, it was demonstrated that the combination of EVs and a sub-inhibitory concentration of aztreonam (0.1 μg/ml) significantly reduced the formation of biofilms by P. aeruginosa on HBEC, and that this effect could be blocked with a let-7b-5p antagomir (FIG. 6). Taken together, these studies demonstrate that let-7b secreted in EVs inhibits biofilm formation by P. aeruginosa.

EV containing let-7b reduces the protein abundance of the biofilm genes NirS and NosZ. Moreover, EV significantly reduced NosZ and NirS in six clinical isolates of P. aeruginosa (Data not shown).

### Example 4. EV and let-7b Increases P. aeruginosa Sensitivity to β-lactam Antibiotics by Targeting β-lactamases

Several β-lactamases are targets of let-7b. The main mechanism of resistance to β-lactam antibiotics like aztreonam (ATM) and carbenicillin (CAR) in Gram-negative bacteria is expression of β-lactamases, which are enzymes that hydrolyze and inactivate β-lactams¹⁶. All four β-lactamases (PA14_72760, PA14_26350, PA14_00690, and PA14_17570) that were detected in proteomics experiment were reduced ˜20% by exposure to EV.

Studies were conducted to determine if EV increase the sensitivity of P. aeruginosa to the β-lactam antibiotic aztreonam (ATM). Planktonic growth curve assays in the presence or absence of EV (5×10⁹/ml) was performed and the minimum inhibitory concentration (MIC) and non-inhibitory concentration (NIC) were calculated by measuring the OD600 to assess planktonic growth, as previously described^{42, 53}.

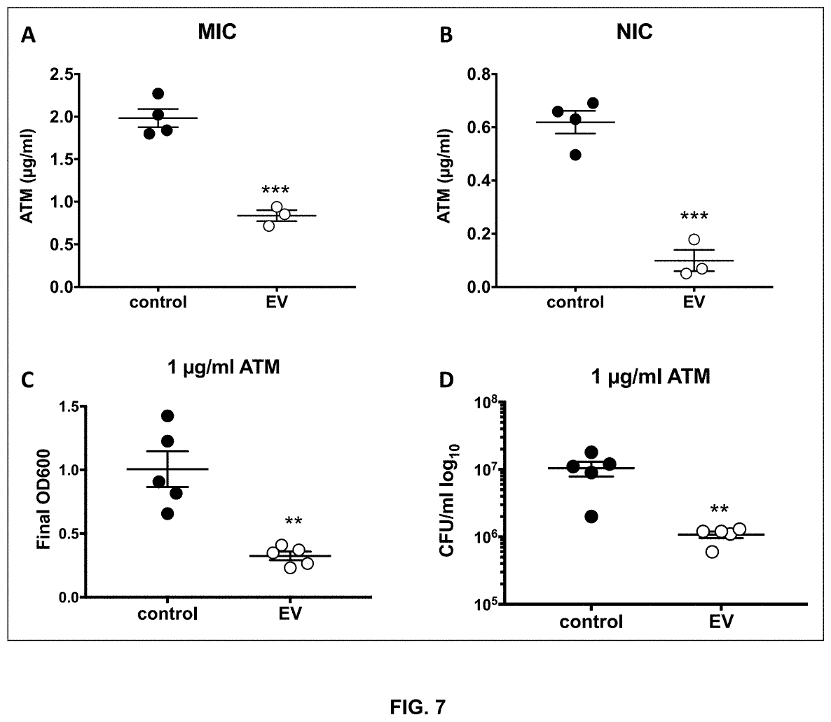

EV decreased both the MIC and NIC for ATM. EV increase the susceptibility of P. aeruginosa to β-lactam antibiotics (FIG. 7A/B). Moreover, in the presence of 1 μg/ml ATM (one-half the MIC of PA14), EV induced a significant reduction in planktonic P. aeruginosa (FIG. 7C). Moreover, in the presence of 1 μg/ml ATM, EV induced a significant reduction in P. aeruginosa colony forming units (CFU) compared to P. aeruginosa not exposed to EV (FIG. 7D). This suggests that OD600 measurements are a good proxy for the number of live bacteria in these experiments.

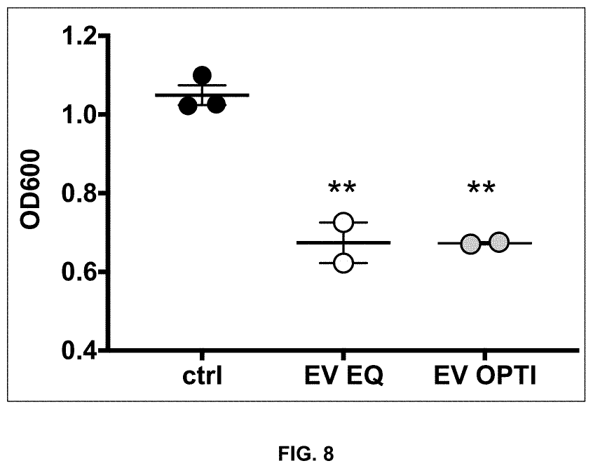

To determine if these results are independent of the EV isolation method, the effect of EV isolated by either ExoQuick-TC® (EQ) or OptiPrep TM gradient ultracentrifugation (OPTI) on planktonic growth of P. aeruginosa in the presence of ATM (1 μg/ml) was compared. It was found that there was no significant difference in the ability of EV isolated by either method to reduce P. aeruginosa planktonic growth in the presence of ATM (FIG. 8).

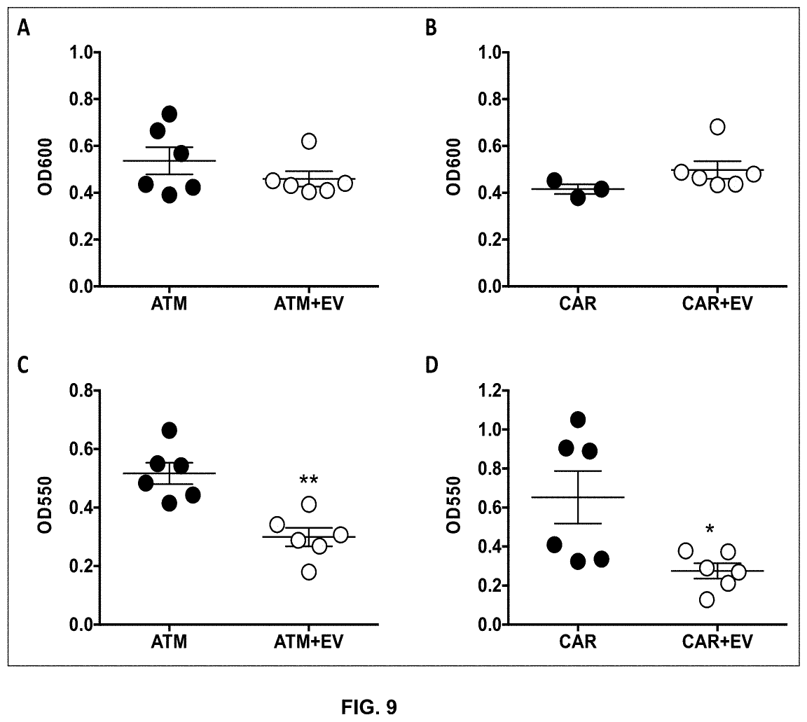

Studies were conducted to determine if EV increase the ability of 3-lactam antibiotics to inhibit planktonic growth and biofilm formation by P. aeruginosa using the crystal violet biofilm plate assay⁶⁴. The subthreshold concentration of ATM (0.1 μg/ml) and CAR (5 μg/ml) for biofilm formation by P. aeruginosa in the absence of EV was determined. At these concentrations of antibiotics, EV did not have a significant effect on planktonic bacteria in biofilm plates after 24 h incubation (FIG. 9A/B), yet significantly reduced the formation of biofilms in the presence of ATM (FIG. 9C) and CAR (FIG. 9D).

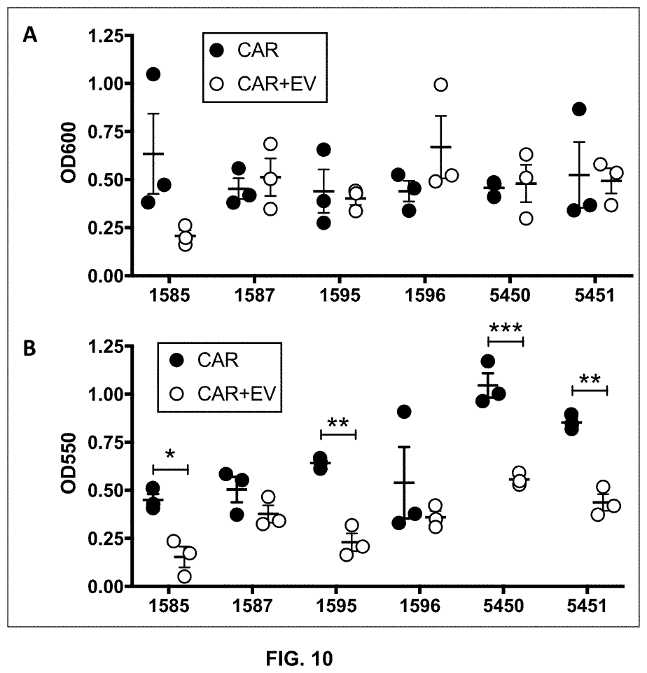

Studies were conducted to assess the ability of EV to reduce biofilm formation by 6 clinical isolates of P. aeruginosa using a concentration of CAR for each isolate that did not affect biofilm formation in the absence of EV. EV did not affect planktonic growth in biofilm plates in the presence of CAR in any of the six clinical isolates (FIG. 10A). For four of the six clinical isolates (1585, 1595, 5450 and 5451), EV significantly decreased biofilm formation in the presence of CAR (FIG. 10B), demonstrating that the effect of EVs to prevent biofilm formation is clinically relevant and not limited to the strain PA14.

### Example 5 NPs, Liposomes and/or MSC EV can be used to Deliver let-7b in Combination with Front Line Antibiotics to Reduce Antibiotic Resistant, Chronic Lung Infections by P. aeruginosa

Let-7b in EV secreted by HBEC in combination with front line antibiotics kills P. aeruginosa more effectively than antibiotics or let-7b alone. A variety of artificial vesicles may be used, including but not limited to nanoparticles that permeate mucus in the lung, as well as liposomes and EV secreted by MSC loaded with let-7b and antibiotics to kill P. aeruginosa more effectively and reduce biofilm formation. EV is secreted by MSC and electroporation is used to load MSC EV with let-7b and antibiotics. MSC EV may lung inflammation in a variety of diseases. NPs, liposomes and MSC EV loaded with tobramycin, ciprofloxacin or aztreonam may kill planktonic P. aeruginosa and well as inhibit biofilm formation and disrupt antibiotic resistant P. aeruginosa biofilms. NPs, liposomes and MSC EV loaded with tobramycin, ciprofloxacin or aztreonam may eliminate P. aeruginosa infections in a mouse model of lung infections^{27, 41, 43-47, 49, 55, 56}.

### Example 6 EVs Repress Aztreonam-Induced Proteins as well as Proteins Involved in Biofilm Formation

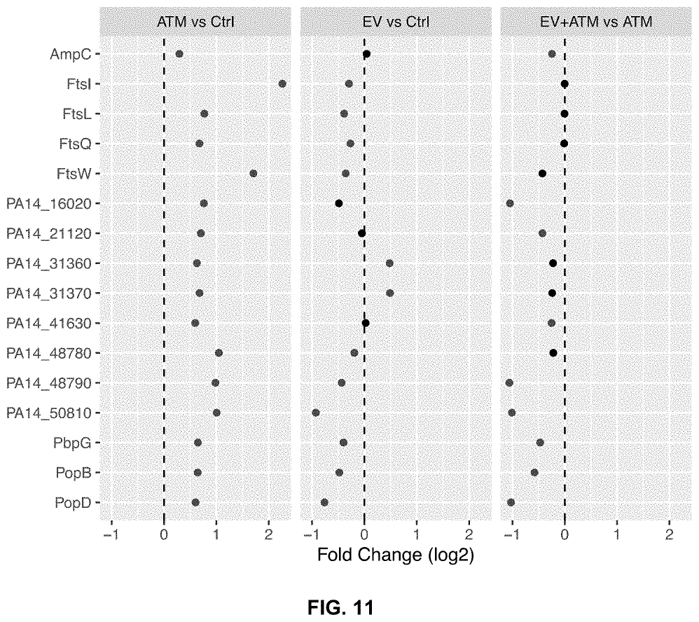

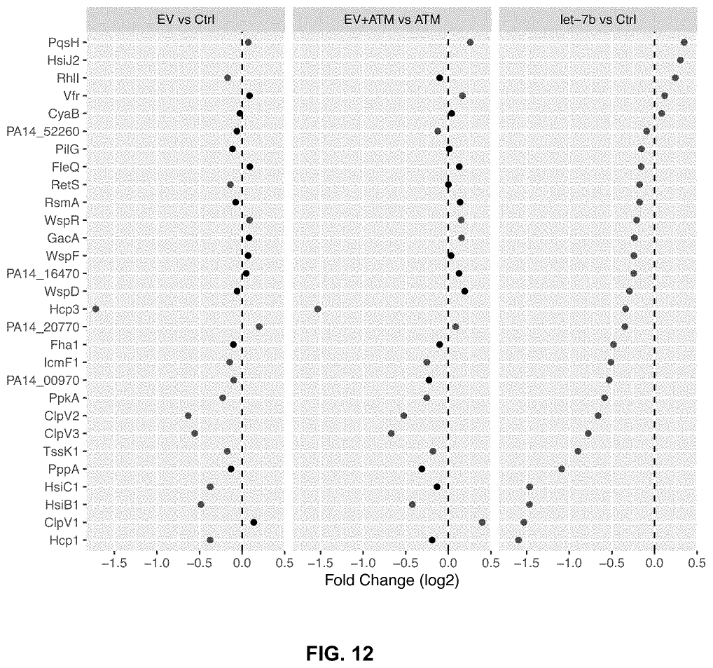

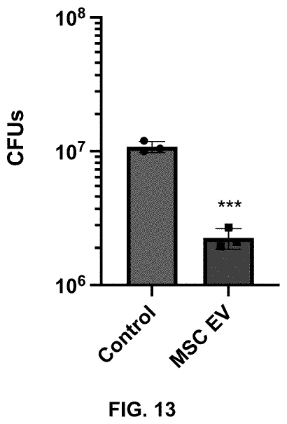

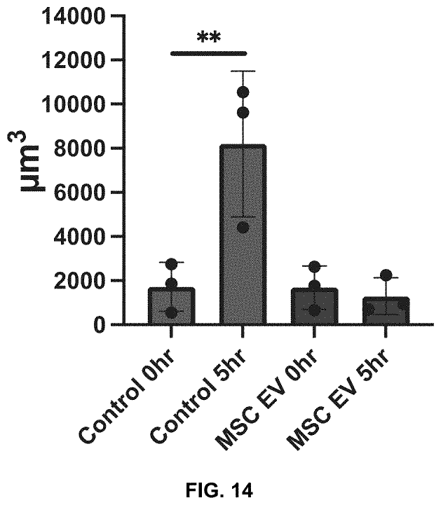

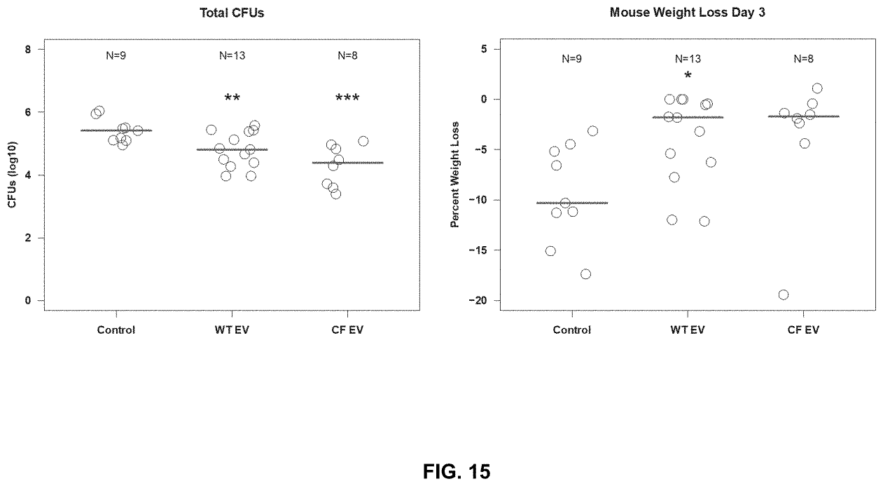

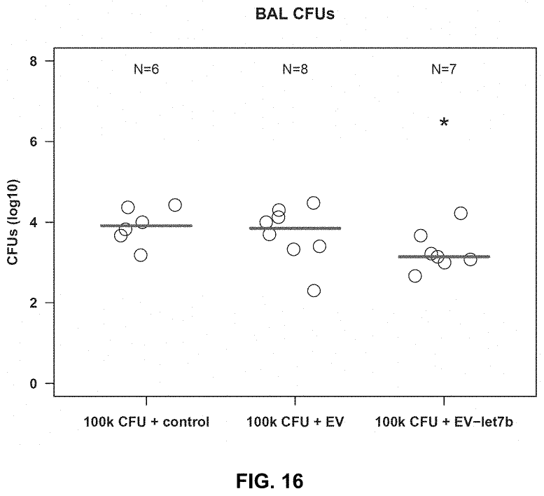

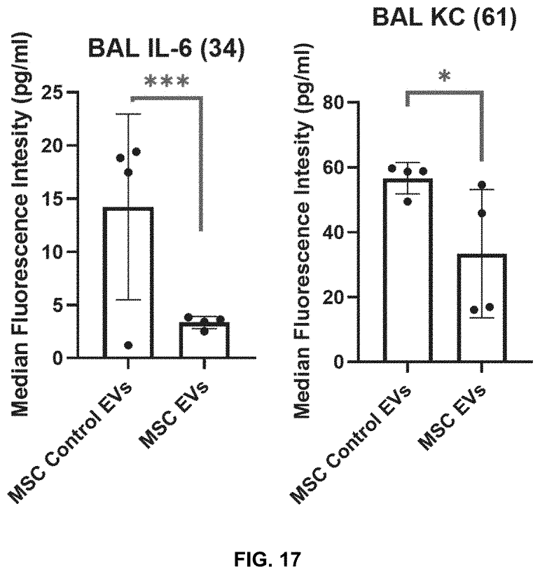

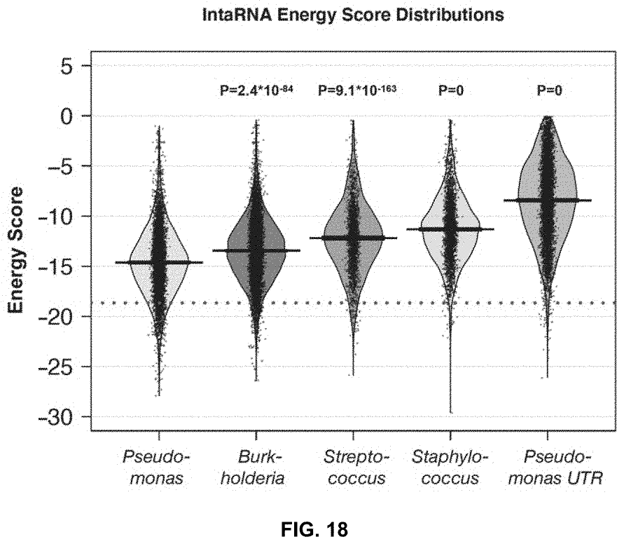

To investigate the cellular mechanisms whereby EVs decrease the MIC and NIC for aztreonam and increase the ability of aztreonam to reduce biofilm formation, an unbiased proteomics analysis was conducted of P. aeruginosa exposed to vehicle or to EVs for 16 hours in the presence or absence of 0.1 μg/ml aztreonam. The beta-lactamase AmpC, which was a predicted let-7b target with a good IntaRNA energy score in P. aeruginosa was significantly induced (p<0.05) by aztreonam and significantly repressed (p<0.05) by the combination of EVs and aztreonam compared to aztreonam alone (FIG. 11). In addition, 15 proteins were significantly induced by aztreonam by at least 50% (p<0.05 and log 2 fold change>0.58). Eight of these aztreonam-induced proteins were significantly repressed (p<0.05) in the presence of EVs and aztreonam compared to aztreonam alone (FIG. 11). Four additional aztreonam-induced proteins showed a tendency to be repressed in the presence of EVs, but this trend did not reach statistical significance. According to the comprehensive antibiotic resistance database (CARD)57, eight of the 15 aztreonam-induced proteins have homology to beta-lactam resistance proteins in other bacteria. Most notably, the hypothetical proteins PA14_48790 and PA14_16020, which were significantly induced by aztreonam and significantly repressed by the combination of EVs and aztreonam, are strong candidates for putative beta-lactamases based on protein sequence homology. Moreover, PA14_15130, another putative beta-lactamase, was significantly reduced in the presence of EVs and aztreonam compared to aztreonam alone. Kyoto Encyclopedia of Genes and Genomes (KEGG) pathway activation analysis of the EV-induced fold changes in protein abundance revealed biofilm formation as the only KEGG pathway predicted to be significantly down-regulated by EVs. This prediction is consistent with our observed phenotype of EV inhibition of biofilm formation. Seven proteins on the KEGG pathway “Biofilm Formation” (Hcp3, IcmF1, PpkA, ClpV2, ClpV3, TssK1 and HsiB1) were significantly (p<0.05) repressed by EVs compared to control samples in the absence of antibiotics as well as EVs and aztreonam versus aztreonam alone (FIG. 12). Additional experiments, described below, demonstrate directly that let-7b-5p itself is sufficient to suppress proteins essential for biofilm formation.

### Example 7 Human Bone Marrow Derived Mesenchymal Stromal Cell (MSC) EV Reduced Colony Forming Units (CFU) Counts in Pseudomonas Biofilms Grown on WT-HBEC

To determine if human bone marrow derived mesenchymal stromal cell (MSC) EV reduced Colony Forming Units (CFU) counts in Pseudomonas biofilms grown on WT-HBEC, Pseudomonas was exposed overnight to MSC EV and one-half the minimal inhibitory concentration (MIC) for aztreonam and then applied to the apical surface of HBEC for 5 hours, at which time CFU were measured as described. Control Pseudomonas was exposed overnight to one-half the minimal inhibitory concentration for aztreonam and then applied to the apical surface of HBEC for 6 hours, at which time CFU were measured as described 22. There is a highly significant difference between 5 h control and 5 h MSC EV (*** p<2e-16). N=3 HBEC donors. Human bone marrow derived MSCs were obtained from the NIH Production Assistance in Cell Therapy (PACT) Program at the University of Minnesota. As shown in FIG. 13, human bone marrow derived mesenchymal stromal cell (MSC) EV reduced Colony Forming Units (CFU) counts in Pseudomonas biofilms grown on WT-HBEC.

### Example 8 Human Bone Marrow Derived Mesenchymal Stromal Cell (MSC) EV Block the Formation of Biofilms on Primary HBEC

To determine whether MSC EV block the formation of biofilms on primary HBEC, Pseudomonas was exposed overnight to MSC EV and one-half the minimal inhibitory concentration (MIC) for aztreonam and then applied to the apical surface of HBEC for 5 hours, at which time images were taken and analyzed as described²². Control Pseudomonas was exposed overnight to one-half the minimal inhibitory concentration for aztreonam and then applied to the apical surface of HBEC for 6 hours, at which time images were taken and analyzed as described²². There is a highly significant difference for the biofilm volumes of 5 h control vs 0 h control (**p=0.00469), whereas there was no significant difference between 0 h MSC EV and 0 h control or between 5 h MSC EV versus 0 h control. There also was a significant difference between 5 h control and 5 h MSC EV (p=0.01734). N=3 HBEC donors. As shown in FIG. 14, MSC EV block the formation of biofilms on primary HBEC.

### Example 9 HBEC EV Reduce Pseudomonas in a CF Mouse Model of Lung Infection

To determine whether HBEC EV reduce Pseudomonas in a CF mouse model of lung infection, EV were isolated from WT and CF HBEC, and both male and female Cftr gut corrected mouse B6.129 Cftr^{tm1Kth} Tg(FABPCFTR)1Jaw/Cwr at approximately 10 weeks of age were infected with 100,000 CFU of PA14 simultaneously with EV (109/ml) instilled using the Polydiagnostic microendoscopy system. None of the mice received antibiotics. Bronchoalveolar fluid (BAL) and lungs were harvested three days after treatment, homogenized, and cultured to count CFU of Pseudomonas. Control was media not exposed to HBEC but run thru the EV isolation procedure to account for any possible effect of the media and/or the ExoQuick EV isolation column that may have affected CFU or other measured parameters. Both WT EV and CF EVs significantly reduced Pseudomonas CFU (*P<0.05, **P<0.01, ***P<0.001) and prevented weight loss due to infection (FIG. 15). These data provide proof of principle that EV suppress Pseudomonas in a CF mouse model of infection.

### Example 10 Mesenchymal Stromal Cell (MSC) EV Reduce Pseudomonas in a CF Mouse Model of Lung Infection

To investigate whether mesenchymal stromal cell (MSC) EV reduce Pseudomonas in a CF mouse model of lung infection, EV were isolated from human bone marrow MSC and both male and female Cftr gut corrected mouse B6.129 Cftr^{tm1Kth} Tg(FABPCFTR)1Jaw/Cwr at approximately 10 weeks of age were infected with 100,000 CFU of PA14 simultaneously with MSC EV (109/ml) instilled using the Polydiagnostic microendoscopy system. None of the mice received antibiotics. Bronchoalveolar fluid (BAL) and lungs were harvested, homogenized, and cultured to count CFU of Pseudomonas. Control was media not exposed to MSC but run through the EV isolation procedure to account for any possible effect of the media and/or the ExoQuick EV isolation column that may have affected CFU or other measured parameters. Only the MSC EV transfected with let-7b decreased Pseudomonas CFU compared to control (*P<0.05). Because control MSC EV contain very little let-7b, as determined by PCR (32-fold less that HBEC EV) our working hypothesis is the paucity of let-7b in control MSC EV was the reason that MSC EV did not reduce CFU. As shown in FIG. 16, mesenchymal stromal cell (MSC) EV reduce Pseudomonas in a CF mouse model of lung infection. These data provide proof of principle that MSC EV containing let-7b suppress Pseudomonas in a CF mouse model of infection.

### Example 11 MSC EV Reduced Inflammation in CF Mouse Lung

In order to determine if MSC EV reduce inflammation in CF mouse lung, both male and female Cftr gut corrected mouse B6.129 Cftr^{tm1Kth} Tg(FABPCFTR)1Jaw/Cwr at approximately 10 weeks of age were infected with 100,000 CFU of PA14 simultaneously with MSC EV (10⁹/ml) instilled using the Polydiagnostic microendoscopy system. Bronchoalveolar fluid (BAL) was isolated for analysis of cytokines. Control was media not exposed to MSC but run thru the EV isolation procedure to account for any possible effect of the media and/or the ExoQuick EV isolation column that may have affected CFU or other measured parameters. Compared to control, MSC EV decreased IL-6 (P<0.001) and KC (homolog to human IL-8) (P<0.05). As shown in FIG. 17, MSC EV reduced inflammation in CF mouse lung. These experiments confirm published studies demonstrating that MSC EV are anti-inflammatory²³⁻²⁵. These data provide proof of principle that MSC EV suppress inflammation due to Pseudomonas infection in a CF mouse model of infection.

### Example 12 Targets of let-7b in Other Lung Pathogens

FIG. 18 shows the predicted targets of let-7b in other lung pathogens. These bioinformatic predictions suggest that let-7b also targets key genes involved in biofilm formation and antibiotic resistance in Burkholderia and Streptococcus, which also cause major lung infections in people with CF. Figure presents IntaRNA targeting predictions for let-7b-5p. Violin plot of the distribution of energy scores from IntaRNA predictions for let-7b-5p targeted genes in Pseudomonas aeruginosa strain 179 PA14 (light green), Burkholderia cenocepacia strain J2315 (blue), Streptococcus pneumoniae 180 strain R6 (magenta), Staphylococcus aureus strain COL (yellow) as well as Pseudomonas aeruginosa intergenic regions (dark green). A lower energy score is associated with a higher likelihood of targeting. The mean IntaRNA energy score for Pseudomonas genes was significantly lower than those of Burkholderia, Streptococcus, Staphylococcus or Pseudomonas untranslated regions (UTR). The red dotted line indicates the top 10% energy score cutoff for Pseudomonas genes of −18.66. Horizontal lines indicate means±95% confidence intervals. Linear models in R were used to calculate P values; N=5942 Pseudomonas genes; N=7344 187 Burkholderia genes; N=2038 Streptococcus genes; N=2683 Staphylococcus genes; N=6064 188 Pseudomonas intergenic regions.

### REFERENCES

The following references are incorporated herein in their entirety:
1. Centers for Disease Control and Prevention. Antibiotic Resistance Threats in the United States;. Atlanta, GA, USA: U.S. Department of Health and Human Services, CDC, 2019.
2. Williams B J, Dehnbostel J, Blackwell T S. Pseudomonas aeruginosa: host defence in lung diseases. Respirology. 2010; 15(7):1037-56. Epub 2010 Aug. 21. doi: 10.1111/j.1440-1843.2010.01819.x. PubMed PMID: 20723140.
3. Ballok A E, O'Toole G A. Pouring salt on a wound: Pseudomonas aeruginosa virulence factors alter Na+ and Cl− flux in the lung. J Bacteriol. 2013; 195(18):4013-9. Epub 2013 Jul. 10. doi: 10.1128/JB.00339-13. PubMed PMID: 23836869; PMCID: PMC3754746.
4. Konstan M W, Wagener J S, Vandevanter D R, Pasta D J, Yegin A, Rasouliyan L, Morgan W J. Risk factors for rate of decline in FEV1 in adults with cystic fibrosis. J Cyst Fibros. 2012; 11(5):405-11. Epub 2012 May 9. doi: 10.1016/j.jcf.2012.03.009. PubMed PMID: 22561369; PMCID: PMC: 4086189.
5. Novosad S A, Barker A F. Chronic obstructive pulmonary disease and bronchiectasis. Curr Opin Pulm Med. 2013; 19(2):133-9. Epub 2013 Jan. 5. doi: 10.1097/MCP.0b013e32835d8312. PubMed PMID: 23287285; PMCID: PMID: 23287285
6. Lieberman D, Lieberman D. Pseudomonal infections in patients with COPD: epidemiology and management. Am J Respir Med. 2003; 2(6):459-68. Epub 2004 Jan. 15. doi: 10.1007/bf03256673. PubMed PMID: 14719985.
7. Mulcahy L R, Isabella V M, Lewis K. Pseudomonas aeruginosa biofilms in disease. Microb Ecol. 2014;68(1):1-12. Epub 2013 Oct. 8. doi: 10.1007/s00248-013-0297-x. PubMed PMID: 24096885; PMCID: PMC3977026.
8. Sethi S. Infection as a comorbidity of COPD. Eur Respir J. 2010; 35(6):1209-15. Epub 2010 Jun. 2. doi: 10.1183/09031936.00081409. PubMed PMID: 20513910.
9. Stanton B A. Effects of Pseudomonas aeruginosa on CFTR chloride secretion and the host immune response. Am J Physiol Cell Physiol. 2017; 312(4):C357-C66. Epub 2017 Jan. 27. doi: 10.1152/ajpcell.00373.2016. PubMed PMID: 28122735; PMCID: PMC5407021.
10. Davies J C, Martin I. New anti-pseudomonal agents for cystic fibrosis—still needed in the era of small molecule CFTR modulators? Expert Opin Pharmacother. 2018; 19(12):1327-36. Epub 2018 Aug. 14. doi: 10.1080/14656566.2018.1505864. PubMed PMID: 30101632; PMCID: PMID: 30101632.
11. Pittman J E, Cutting G, Davis S D, Ferkol T, Boucher R. Cystic fibrosis: NHLBI Workshop on the Primary Prevention of Chronic Lung Diseases. Ann Am Thorac Soc. 2014; 11 Suppl 3:S161-8. Epub 2014 Apr. 24. doi: 10.1513/AnnalsATS.201312-444LD. PubMed PMID: 24754825; PMCID: PMC5473656.
12. Collawn J F, Matalon S. CFTR and lung homeostasis. Am J Physiol Lung Cell Mol Physiol. 2014; 307(12):L917-23. Epub 2014 Nov. 9. doi: 10.1152/ajplung.00326.2014. PubMed PMID: 25381027; PMCID: PMC4269691.
13. Cohen T S, Prince A. Cystic fibrosis: a mucosal immunodeficiency syndrome. Nat Med. 2012; 18(4):509-19. Epub 2012 Apr. 7. doi: 10.1038/nm.2715. PubMed PMID: 22481418; PMCID: PMC3577071.
14. de la Fuente-Nunez C, Reffuveille F, Fairfull-Smith K E, Hancock R E. Effect of nitroxides on swarming motility and biofilm formation, multicellular behaviors in Pseudomonas aeruginosa. Antimicrob Agents Chemother. 2013; 57(10):4877-81. Epub 2013 Jul. 24. doi: 10.1128/AAC.01381-13. PubMed PMID: 23877682; PMCID: PMC3811460.
15. Kang D, Kirienko N V. Interdependence between iron acquisition and biofilm formation in Pseudomonas aeruginosa. J Microbiol. 2018; 56(7):449-57. Epub 2018 Jun. 28. doi: 10.1007/s12275-018-8114-3. PubMed PMID: 29948830; PMCID: PMC: 6221862.
16. Rice L B. Mechanisms of resistance and clinical relevance of resistance to beta-lactams, glycopeptides, and fluoroquinolones. Mayo Clin Proc. 2012; 87(2):198-208. Epub 2012/02/07. doi: 10.1016/j.mayocp.2011.12.003. PubMed PMID: 22305032; PMCID: PMC3498059.
17. Dreier J, Ruggerone P. Interaction of antibacterial compounds with RND efflux pumps in Pseudomonas aeruginosa. Front Microbiol. 2015; 6:660. Epub 2015 Jul. 29. doi: 10.3389/fmicb.2015.00660. PubMed PMID: 26217310; PMCID: PMC4495556.
18. Puzari M, Chetia P. RND efflux pump mediated antibiotic resistance in Gram-negative bacteria Escherichia coli and Pseudomonas aeruginosa: a major issue worldwide. World J Microbiol Biotechnol. 2017; 33(2):24. Epub 2017 Jan. 4. doi: 10.1007/s11274-016-2190-5. PubMed PMID: 28044273; PMCID: PMID: 28044273
19. Rutter W C, Burgess D R, Burgess D S. Increasing Incidence of Multidrug Resistance Among Cystic Fibrosis Respiratory Bacterial Isolates. Microb Drug Resist. 2017; 23(1):51-5. Epub 2016 Jun. 22. doi: 10.1089/mdr.2016.0048. PubMed PMID: 27326758.
20. Kidd T J, Canton R, Ekkelenkamp M, Johansen H K, Gilligan P, LiPuma J J, Bell S C, Elborn J S, Flume P A, VanDevanter D R, Waters V J, Antimicrobial Resistance in Cystic Fibrosis International Working G. Defining antimicrobial resistance in cystic fibrosis. J Cyst Fibros. 2018; 17(6):696-704. Epub 2018 Sep. 30. doi: 10.1016/j.jcf.2018.08.014. PubMed PMID: 30266518.
21. Mulani M S, Kamble E E, Kumkar S N, Tawre M S, Pardesi K R. Emerging Strategies to Combat ESKAPE Pathogens in the Era of Antimicrobial Resistance: A Review. Front Microbiol. 2019; 10:539. Epub 2019 Apr. 17. doi: 10.3389/fmicb.2019.00539. PubMed PMID: 30988669; PMCID: PMC6452778.
22. Koeppen K, Nymon, A., Barnaby, R., Bashor, L., Li, Z., Hampton, T. H., Liefeld, A .E., Kolling, F. W., LaCroix, I. S., Gerber, S. A., Hogan, D. A., Kasetty, S., Nadell, C. and B. A. Stanton. Let-7b-5p in vesicles secreted by human airway cells reduces biofilm formation and increases antibiotic sensitivity of P. aeruginosa. Proc Natl Acad Sci USA. 2021; 118(28):e2105370118.
.doi: doi: 10.1073/pnas.2105370118.
23. Lu Y, Wang L, Zhang M, Chen Z. Mesenchymal Stem Cell-Derived Small Extracellular Vesicles: A Novel Approach for Kidney Disease Treatment. Int J Nanomedicine. 2022; 17:3603-18. Epub 2022 Aug. 13. doi: 10.2147/IJN.S372254. PubMed PMID: 35990308; PMCID: PMC9386173.
24. Sang L, Guo X, Fan H, Shi J, Hou S, Lv Q. Mesenchymal Stem Cell-Derived Extracellular Vesicles as Idiopathic Pulmonary Fibrosis Microenvironment Targeted Delivery. Cells. 2022; 11(15). Epub 2022 Jul. 28. doi: 10.3390/cells11152322. PubMed PMID: 35954166; PMCID: PMC9367455.
25. Abbaszadeh H, Ghorbani F, Abbaspour-Aghdam S, Kamrani A, Valizadeh H, Nadiri M, Sadeghi A, Shamsasenjan K, Jadidi-Niaragh F, Roshangar L, Ahmadi M. Chronic obstructive pulmonary disease and asthma: mesenchymal stem cells and their extracellular vesicles as potential therapeutic tools. Stem Cell Res Ther. 2022; 13(1):262. Epub 2022 Jun. 20. doi: 10.1186/s13287-022-02938-5. PubMed PMID: 35725505; PMCID: PMC9208161.
26. Goodale B C, Rayack E J, Stanton B A. Arsenic alters transcriptional responses to Pseudomonas aeruginosa infection and decreases antimicrobial defense of human airway epithelial cells. Toxicol Appl Pharmacol. 2017; 331:154-63. Epub 2017 Jun. 20. doi: 10.1016/j.taap.2017.06.010. PubMed PMID: 28625800; PMCID: PMC5568502.
27. Koeppen K, Hampton T H, Jarek M, Scharfe M, Gerber S A, Mielcarz D W, Demers E G, Dolben E L, Hammond J H, Hogan D A, Stanton B A. A Novel Mechanism of Host-Pathogen Interaction through sRNA in Bacterial Outer Membrane Vesicles. PLoS Pathog. 2016; 12(6):e1005672. Epub 2016 Jun. 15. doi: 10.1371/journal.ppat.1005672. PubMed PMID: 27295279; PMCID: PMC4905634.
28. Baglio S R, Rooijers K, Koppers-Lalic D, Verweij F J, Perez Lanzon M, Zini N, Naaijkens B, Perut F, Niessen H W, Baldini N, Pegtel D M. Human bone marrow- and adipose-mesenchymal stem cells secrete exosomes enriched in distinctive miRNA and tRNA species. Stem Cell Res Ther. 2015; 6:127. Epub 2015 Jul. 2. doi: 10.1186/s13287-015-0116-z. PubMed PMID: 26129847; PMCID: PMC4529699.
29. Vojtech L, Woo S, Hughes S, Levy C, Ballweber L, Sauteraud R P, Strobl J, Westerberg K, Gottardo R, Tewari M, Hladik F. Exosomes in human semen carry a distinctive repertoire of small non-coding RNAs with potential regulatory functions. Nucleic Acids Res. 2014; 42(11):7290-304. Epub 2014 May. 20. doi: 10.1093/nar/gku347. PubMed PMID: 24838567; PMCID: PMC4066774.
30. Quek C, Bellingham S A, Jung C H, Scicluna B J, Shambrook M C, Sharples R A, Cheng L, Hill A F. Defining the purity of exosomes required for diagnostic profiling of small RNA suitable for biomarker discovery. RNA Biol. 2017; 14(2):245-58. Epub 2016 Dec. 23. doi: 10.1080/15476286.2016.1270005. PubMed PMID: 28005467; PMCID: PMC5324750.
31. Choi J W, Um J H, Cho J H, Lee H J. Tiny RNAs and their voyage via extracellular vesicles: Secretion of bacterial small RNA and eukaryotic microRNA. Exp Biol Med (Maywood). 2017; 242(15):1475-81. Epub 2017 Jul. 26. doi: 10.1177/1535370217723166. PubMed PMID: 28741379; PMCID: PMC5648287.
32. Garcia-Silva M R, Cabrera-Cabrera F, Guida M C, Cayota A. Hints of tRNA-Derived Small RNAs Role in RNA Silencing Mechanisms. Genes (Basel). 2012; 3(4):603-14. Epub 2012 Jan. 1. doi: 10.3390/genes3040603. PubMed PMID: 24705078; PMCID: PMC3899978.
33. Lee Y S, Shibata Y, Malhotra A, Dutta A. A novel class of small RNAs: tRNA-derived RNA fragments (tRFs). Genes Dev. 2009; 23(22):2639-49. Epub 2009 Nov. 26. doi: 10.1101/gad.1837609. PubMed PMID: 19933153; PMCID: PMC2779758.
34. Kumar P, Anaya J, Mudunuri S B, Dutta A. Meta-analysis of tRNA derived RNA fragments reveals that they are evolutionarily conserved and associate with AGO proteins to recognize specific RNA targets. BMC Biol. 2014; 12:78. Epub 2014 Oct. 2. doi: 10.1186/s12915-014-0078-0. PubMed PMID: 25270025; PMCID: PMC4203973.
35. Bomberger J M, Maceachran D P, Coutermarsh B A, Ye S, O'Toole G A, Stanton B A. Long-distance delivery of bacterial virulence factors by Pseudomonas aeruginosa outer membrane vesicles. PLoS Pathog. 2009; 5(4):e1000382. Epub 2009 Apr. 11. doi: 10.1371/journal.ppat.1000382. PubMed PMID: 19360133; PMCID: PMC2661024.
36. Guha A, Choudhury A, Unni B G, Roy M K. Effect of outer-membrane permeabilizers on the activity of antibiotics and plant extracts against Pseudomonas aeruginosa. Folia Microbiol (Praha). 2002; 47(4):379-84. Epub 2002 Nov. 9. doi: 10.1007/bf02818694. PubMed PMID: 12422514.
37. Briers Y, Walmagh M, Lavigne R. Use of bacteriophage endolysin EL188 and outer membrane permeabilizers against Pseudomonas aeruginosa. J Appl Microbiol. 2011; 110(3):778-85. Epub 2011 Jan. 19. doi: 10.1111/j.1365-2672.2010.04931.x. PubMed PMID: 21241420.
38. Sekiya H, Mima T, Morita Y, Kuroda T, Mizushima T, Tsuchiya T. Functional cloning and characterization of a multidrug efflux pump, mexHI-opmD, from a Pseudomonas aeruginosa mutant. Antimicrob Agents Chemother. 2003; 47(9):2990-2. Epub 2003 Aug. 26. doi: 10.1128/aac.47.9.2990-2992.2003. PubMed PMID: 12937010; PMCID: PMC182609.
39. Wolloscheck D, Krishnamoorthy G, Nguyen J, Zgurskaya H I. Kinetic Control of Quorum Sensing in Pseudomonas aeruginosa by Multidrug Efflux Pumps. ACS Infect Dis. 2018; 4(2):185-95. Epub 2017 Nov. 9. doi: 10.1021/acsinfecdis.7b00160. PubMed PMID: 29115136; PMCID: PMC5807214.
40. Winsor G L, Griffiths E J, Lo R, Dhillon B K, Shay J A, Brinkman F S. Enhanced annotations and features for comparing thousands of Pseudomonas genomes in the Pseudomonas genome database. Nucleic Acids Res. 2016; 44(D1):D646-53. Epub 2015 Nov. 19. doi: 10.1093/nar/gkv1227. PubMed PMID: 26578582; PMCID: PMC4702867.
41. Yu Q, Griffin E F, Moreau-Marquis S, Schwartzman J D, Stanton B A, O'Toole G A. In vitro evaluation of tobramycin and aztreonam versus Pseudomonas aeruginosa biofilms on cystic fibrosis-derived human airway epithelial cells. J Antimicrob Chemother. 2012; 67(11):2673-81. Epub 2012 Jul. 31. doi: 10.1093/jac/dks296. PubMed PMID: 22843834; PMCID: PMC3468082.
42. Malek A A, Wargo M J, Hogan D A. Absence of membrane phosphatidylcholine does not affect virulence and stress tolerance phenotypes in the opportunistic pathogen Pseudomonas aeruginosa. PLoS One. 2012; 7(2):e30829. Epub 2012 Mar. 1. doi: 10.1371/journal.pone.0030829. PubMed PMID: 22363496; PMCID: PMC3281885.
43. Moreau-Marquis S, Redelman C V, Stanton B A, Anderson G G. Co-culture models of Pseudomonas aeruginosa biofilms grown on live human airway cells. J Vis Exp. 2010(44). Epub 2010 Oct. 26. doi: 10.3791/2186. PubMed PMID: 20972407; PMCID: PMC3185622.
44. Moreau-Marquis S, O'Toole G A, Stanton B A. Tobramycin and FDA-approved iron chelators eliminate Pseudomonas aeruginosa biofilms on cystic fibrosis cells. Am J Respir Cell Mol Biol. 2009; 41(3):305-13. Epub 2009 Jan. 27. doi: 10.1165/rcmb.2008-02990C. PubMed PMID: 19168700; PMCID: PMC2742750.
45. Moreau-Marquis S, Bomberger J M, Anderson G G, Swiatecka-Urban A, Ye S, O'Toole G A, Stanton B A. The DeltaF508-CFTR mutation results in increased biofilm formation by Pseudomonas aeruginosa by increasing iron availability. Am J Physiol Lung Cell Mol Physiol. 2008; 295(1):L25-37. Epub 2008 Mar. 25. doi: 10.1152/ajplung.00391.2007. PubMed PMID: 18359885; PMCID: PMC2494796.
46. Anderson G G, Moreau-Marquis S, Stanton B A, O'Toole G A. In vitro analysis of tobramycin-treated Pseudomonas aeruginosa biofilms on cystic fibrosis-derived airway epithelial cells. Infect Immun. 2008; 76(4):1423-33. Epub 2008 Jan. 24. doi: 10.1128/IAI.01373-07. PubMed PMID: 18212077; PMCID: PMC2292855.
47. Moreau-Marquis S, Coutermarsh B, Stanton B A. Combination of hypothiocyanite and lactoferrin (ALX-109) enhances the ability of tobramycin and aztreonam to eliminate Pseudomonas aeruginosa biofilms growing on cystic fibrosis airway epithelial cells. J Antimicrob Chemother. 2015; 70(1):160-6. Epub 2014 Sep. 13. doi: 10.1093/jac/dku357. PubMed PMID: 25213272; PMCID: PMC4267504.
48. Heussler G E, Cady K C, Koeppen K, Bhuju S, Stanton B A, O'Toole G A. Clustered Regularly Interspaced Short Palindromic Repeat-Dependent, Biofilm-Specific Death of Pseudomonas aeruginosa Mediated by Increased Expression of Phage-Related Genes. MBio. 2015; 6(3):e00129-15. Epub 2015 May 15. doi: 10.1128/mBio.00129-15. PubMed PMID: 25968642; PMCID: PMC4436051.
49. Jackson A A, Gross M J, Daniels E F, Hampton T H, Hammond J H, Vallet-Gely I, Dove S L, Stanton B A, Hogan D A. Anr and its activation by PIcH activity in Pseudomonas aeruginosa host colonization and virulence. J Bacteriol. 2013; 195(13):3093-104. Epub 2013 May 15. doi: 10.1128/JB.02169-12. PubMed PMID: 23667230; PMCID: PMC3697539.
50. Barnaby R, Koeppen K, Stanton B A. Cyclodextrins reduce the ability of Pseudomonas aeruginosa outer-membrane vesicles to reduce CFTR Cl(−) secretion. Am J Physiol Lung Cell Mol Physiol. 2019; 316(1):L206-L15. Epub 2018 Oct. 26. doi: 10.1152/ajplung.00316.2018. PubMed PMID: 30358440; PMCID: PMC6383504.
51. Mann M, Wright P R, Backofen R. IntaRNA 2.0: enhanced and customizable prediction of RNA-RNA interactions. Nucleic Acids Res. 2017; 45(W1):W435-W9. Epub 2017 May 5. doi: 10.1093/nar/gkx279. PubMed PMID: 28472523; PMCID: PMC5570192.
52. Busch A, Richter A S, Backofen R. IntaRNA: efficient prediction of bacterial sRNA targets incorporating target site accessibility and seed regions. Bioinformatics. 2008; 24(24):2849-56. Epub 2008 Oct. 23. doi: 10.1093/bioinformatics/btn544. PubMed PMID: 18940824; PMCID: PMC2639303.
53. Lambert R J, Pearson J. Susceptibility testing: accurate and reproducible minimum inhibitory concentration (MIC) and non-inhibitory concentration (NIC) values. J Appl Microbiol. 2000; 88(5):784-90. Epub 2000 May 3. doi: 10.1046/j.1365-2672.2000.01017.x. PubMed PMID: 10792538.
54. O'Toole G A. Microtiter dish biofilm formation assay. J Vis Exp. 2011(47). Epub 2011 Feb. 11. doi: 10.3791/2437. PubMed PMID: 21307833; PMCID: PMC3182663.
55. Hvorecny K L, Dolben E, Moreau-Marquis S, Hampton T H, Shabaneh T B, Flitter B A, Bahl C D, Bomberger J M, Levy B D, Stanton B A, Hogan D A, Madden D R. An epoxide hydrolase secreted by Pseudomonas aeruginosa decreases mucociliary transport and hinders bacterial clearance from the lung. Am J Physiol Lung Cell Mol Physiol. 2018; 314(1):L150-L6. Epub 2017 Oct. 7. doi: 10.1152/ajplung.00383.2017. PubMed PMID: 28982736; PMCID: PMC5866430.
56. Koeppen K, Barnaby R, Jackson A A, Gerber S A, Hogan D A, Stanton B A. Tobramycin reduces key virulence determinants in the proteome of Pseudomonas aeruginosa outer membrane vesicles. PLoS One. 2019; 14(1):e0211290. Epub 2019 Jan. 27. doi: 10.1371/journal.pone.0211290. PubMed PMID: 30682135; PMCID: PMC6347270.
57. Alcock B P, Raphenya A R, Lau T T Y, Tsang K K, Bouchard M, Edalatmand A, Huynh W, Nguyen A V, Cheng A A, Liu S, Min S Y, Miroshnichenko A, Tran H K, Werfalli R E, Nasir J A, Oloni M, Speicher D J, Florescu A, Singh B, Faltyn M, Hernandez-Koutoucheva A, Sharma A N, Bordeleau E, Pawlowski A C, Zubyk H L, Dooley D, Griffiths E, Maguire F, Winsor G L, Beiko R G, Brinkman F S L, Hsiao W W L, Domselaar G V, McArthur A G. CARD 2020: antibiotic resistome surveillance with the comprehensive antibiotic resistance database. Nucleic Acids Res. 2020; 48(D1):D517-D25. Epub 2019 Oct. 31. doi: 10.1093/nar/gkz935. PubMed PMID: 31665441; PMCID: PMC7145624.

We claim:

1. A composition comprising
a) a particle selected from the group consisting of a liposome, an extracellular vesicle, a solid lipid nanoparticle, and a polymeric nanoparticle;
b) a microRNA (miRNA); and
c) an antibiotic.

2. The composition of claim 1, wherein the particle is loaded with at least one of the miRNA and the antibiotic.

3. The composition of claim 1, wherein the particle is loaded with both the miRNA and the antibiotic.

4. The composition of claim 1, wherein the particle is an extracellular vesicle.

5. The composition of claim 5, wherein the extracellular vesicle is derived from a human epithelial cell.

6. The composition of claim 5, wherein the extracellular vesicle is derived from a mesenchymal stem cell (MSC).

7. The composition of claim 1, wherein the miRNA targets an efflux pump or β-lactamase.

8. The composition of claim 1, wherein the miRNA targets a Resistance-Nodulation-Division (RND) efflux pump.

9. The composition of claim 1, wherein the miRNA targets a mexGHI-OpmD multi-drug efflux pump.

10. The composition of claim 1, wherein the miRNA targets a gene implicated in forming biofilm in Pseudomonas aeruginosa, Burkholderia cenocepacia, Streptococcus pneumoniae, or Staphylococcus aureus.

11. The composition of claim 1, wherein the miRNA reduces protein abundance of at least one gene selected from the group consisting of NarG, NarH, NarI, NirS, NosZ, PvdA, PvdD, PvdH, PvdJ, PvdQ, PhzE1, and PhzE2a.

12. The composition of claim 1, wherein the miRNA is an RNA having a sequence selected from the group consisting of SEQ ID NO:1, SEQ ID NO:2, SEQ ID NO:3, and SEQ ID NO:4.

13. The composition of claim 1, wherein the antibiotic is selected from the group consisting of β-lactam antibiotics, fluoroquinolone antibiotics, and aminoglycoside antibiotics.

14. The composition of claim 1, wherein the antibiotic is selected from the group consisting of aztreonam, tobramycin, carbenicillin, azithromycin, colistin/polymyxin E, gentamicin and ciprofloxacin.

15. The composition of claim 1, wherein the composition is in the form of an inhalable powder, an aerosol, or a spray.

16. The composition of claim 1, wherein the composition is adapted for aerosolized administration.

17. The composition of claim 1, wherein the particle has a diameter ranging from 10 nm to 1,000 nm.

18. A pharmaceutical composition comprising the composition of claim 1 and a pharmaceutically acceptable carrier.

19. A kit comprising the pharmaceutical composition of claim 18 and instructions for use.

20. A method of inhibiting proliferation of biofilm-forming microorganisms, comprising administering a therapeutically effective amount of the composition of claim 1.

21. The method of claim 20, wherein the biofilm-forming microorganisms are selected from the group consisting of Pseudomonas aeruginosa, Burkholderia cenocepacia, Streptococcus pneumoniae, or Staphylococcus aureus.

22. A method of preventing or treating an infection associated with Pseudomonas aeruginosa in a subject in need thereof, comprising administering to the subject a therapeutically effective amount of the composition of claim 1.

23. A method of reducing proliferation, survival, migration, or colony formation ability of a rapidly proliferating cell in a subject, comprising contacting the cell with a therapeutically effective amount of the composition of claim 1.

24. The method of claim 23, wherein the cell is Pseudomonas aeruginosa, Burkholderia cenocepacia, Streptococcus pneumoniae, or Staphylococcus aureus.

25. A method of reducing proliferation, survival, migration, or colony formation ability of a microorganism in a subject in need thereof, comprising contacting the microorganism with a therapeutically effective amount of the composition of claim 1.

26. The method of claim 25, wherein the microorganism is Pseudomonas aeruginosa, Burkholderia cenocepacia, Streptococcus pneumoniae, or Staphylococcus aureus.

27. The method of claim 20, wherein the composition is administered in an aerosolized form.

28. The method of claim 20, wherein the composition is administered by inhalation.

29. A method of preventing or treating an infection associated with Pseudomonas aeruginosa in a subject in need thereof, comprising administering to the subject an extracellular vesicle loaded with let-7b in combination with an antibiotic.

30. The method of claim 28, wherein the infection is a chronic lung infection.

31. The method of claim 28, wherein the infection is a respiratory tract infection.

32. A method of reducing or preventing biofilm formation by Pseudomonas aeruginosa on a living or nonliving surface comprising treating the surface with an effective amount of the composition of claim 1.

33. A method of preventing or treating an infection associated with a microorganism in a subject in need thereof, comprising administering to the subject a therapeutically effective amount of the composition of claim 1.

34. The method of claim 33, wherein the microorganism is Pseudomonas aeruginosa, Burkholderia cenocepacia, Streptococcus pneumoniae, or Staphylococcus aureus.

35. The method of claim 33, wherein the infection is a chronic lung infection.

36. The method of claim 33, wherein the infection is a respiratory tract infection.

37. A method of reducing or preventing biofilm formation by a microorganism on a living or nonliving surface comprising treating the surface with an effective amount of the composition of claim 1.

38. The method of claim 37, wherein the microorganism is Pseudomonas aeruginosa, Burkholderia cenocepacia, Streptococcus pneumoniae, or Staphylococcus aureus.
